(19) United States
(12) Patent Application Publication
(10) Pub. No.: US 2026/0069593 A1
(43) Pub. Date: Mar. 12, 2026

(54) METHODS FOR TREATING CANCER

(71) Applicant: AMGEN INC., Thousand Oaks, CA (US)

(72) Inventors: Igor Ivanovich Rybkin, Thousand Oaks, CA (US); Haby Henary, Moorpark, CA (US); Gataree Ngarmchamnanrith, Los Angeles, CA (US); Neelesh Soman, Oak Park, CA (US); Gregory Friberg, Westlake Village, CA (US); Bhakti Mehta, Thousand Oaks, CA (US)

(21) Appl. No.: 18/872,959

(22) PCT Filed: Jun. 26, 2023
(86) PCT No.: PCT/US2023/026195

§ 371 Date: Dec. 9, 2024

Related U.S. Application Data
(60) Provisional application No. 63/355,695, filed on Jun. 27, 2022.
(60) Provisional application No. 63/395,032, filed on Aug. 4, 2022.

(51) Int. Cl. A61K 31/519; A61K 9/00; A61K 39/00; A61P 35/00; C07K 16/28

(52) U.S. Cl. CPC A61K 31/519; A61K 9/0019; A61K 9/0053; A61P 35/00; C07K 16/2818; C07K 16/2827; A61K 2039/505; A61K 2039/54; A61K 2039/545

(57) ABSTRACT

Provided herein are methods of treating cancer comprising a KRAS G12C mutation in a patient comprising (a) administering to the patient a therapeutically effective amount of sotorasib for 14 to 48 days (“an induction period”), and (b) administering to the patient a therapeutically effective amount of sotorasib and a therapeutically effective amount of an anti-PD 1 antibody or an anti-PD-L1 antibody after the induction period for the duration of a combination period.

### CROSS-REFERENCE TO RELATED APPLICATIONS

This application claims the benefit of priority to U.S. Provisional Patent Application No. 63/355,695, filed Jun. 27, 2022 and U.S. Provisional Patent Application No. 63/395,032, filed Aug. 4, 2022, both of which are herewith incorporated by reference in their entireties for all purposes.

### BACKGROUND

The rat sarcoma (RAS) proto-oncogene has been identified as an oncogenic driver of tumorigenesis in cancers, such as non-small cell lung cancer (NSCLC) and colorectal cancer (CRC). The RAS family consists of 3 closely related genes that express guanosine triphosphate (GTP)-ases responsible for regulating cellular proliferation and survival. The RAS proteins, Kirsten rat sarcoma viral oncogene homolog (KRAS), Harvey rat sarcoma viral oncogene homolog (HRAS), and neuroblastoma RAS viral oncogene homolog (NRAS) can be mutationally activated at codons 12, 13, or 61, leading to human cancers. Different tumor types are associated with mutations in certain isoforms of RAS, with KRAS being the most frequently mutated isoform in most cancers. While the role of KRAS mutations in human cancers has been known for decades, no anti-cancer therapies specifically targeting KRAS mutations have been successfully developed, until recently, largely because the protein had been considered intractable for inhibition by small molecules. Sotorasib, sold in the United States under the name LUMAKRAS®, is the first inhibitor of the RAS GTPase family indicated for the treatment of adult patients with KRAS G12C-mutated locally advanced or metastatic NSCLC, as determined by an U.S. Food and Drug Administration (FDA)-approved test, who have received at least one prior systemic therapy. While sotorasib monotherapy appears to drive substantial anti-tumor activity, combination therapies are desirable to further improve treatment options available to patients.

### SUMMARY

Provided herein are methods of treating cancer comprising a KRAS G12C mutation in a patient comprising (a) administering to the patient a therapeutically effective amount of sotorasib for 14 to 48 days (“an induction period”), and (b) administering to the patient a therapeutically effective amount of sotorasib and a therapeutically effective amount of an anti-PD1 antibody or an anti-PD-L1 antibody after the induction period for the duration of a combination period. In various embodiments, the induction period is 21 days. In various embodiments, the induction period is 42 days. In various embodiments, the combination period is at least 30 days. In various embodiments, the combination period is at least 3 months. In various embodiments, the combination period is at least 6 months. In various embodiments, the combination period is at least 8 months.

In various embodiments, the therapeutically effective amount of sotorasib administered for the duration of the induction period is 960 mg. In various embodiments, the therapeutically effective amount of sotorasib administered for the duration of the induction period is 360 mg. In various embodiments, the therapeutically effective amount of sotorasib administered for the duration of the induction period is 240 mg. In various embodiments, the therapeutically effective amount of sotorasib administered for the duration of the induction period is 120 mg. In various embodiments, the sotorasib is administered once daily during the induction period. In various embodiments, the sotorasib is administered twice daily during the induction period, wherein each dose of sotorasib corresponds to half of the therapeutically effective amount administered during the induction period.

In various embodiments, the therapeutically effective amount of sotorasib administered for the duration of the combination period is 960 mg. In various embodiments, the therapeutically effective amount of sotorasib administered for the duration of the combination period is 360 mg. In various embodiments, the therapeutically effective amount of sotorasib administered for the duration of the combination period is 240 mg. In various embodiments, the therapeutically effective amount of sotorasib administered for the duration of the combination period is 120 mg. In various embodiments, the sotorasib is administered once daily during the combination period. In various embodiments, the sotorasib is administered twice daily during the combination period, wherein each dose of sotorasib corresponds to half of the therapeutically effective amount administered during the combination period.

In various embodiments, the anti-PD-L1 antibody is atezolizumab, avelumab, or durvalumab. In various embodiments, the anti-PD-L1 antibody is atezolizumab. In various embodiments, the anti-PD1 antibody is cemiplimab, dostarlimab, pembrolizumab, or nivolumab. In various embodiments, the anti-PD1 antibody is pembrolizumab. In various embodiments, 200 mg pembrolizumab is administered to the patient via IV once every three weeks during the combination period.

In various embodiments, the patient is administered 360 mg sotorasib orally once daily during each of the induction period and the combination period and 200 mg pembrolizumab via IV once every three weeks during the combination period. In various embodiments, the patient is administered 960 mg sotorasib orally once daily during each of the induction period and the combination period and 200 mg pembrolizumab via IV once every three weeks during the combination period. In various embodiments, the patient is administered 240 mg sotorasib orally once daily during each of the induction period and the combination period and 200 mg pembrolizumab via IV once every three weeks during the combination period. In various embodiments, the patient is administered 120 mg sotorasib orally once daily during each of the induction period and the combination period and 200 mg pembrolizumab via IV once every three weeks during the combination period.

In various embodiments, the cancer exhibits a PD-L1 tumor proportion score (TPS) of 1% or greater. In various embodiments, the cancer exhibits a PD-L1 tumor proportion score (TPS) of 50% or greater. In various embodiments, the cancer exhibits a PD-L1 tumor proportion score (TPS) of 1% to 49%. In various embodiments, the cancer exhibits a PD-L1 tumor proportion score (TPS) of less than 1%. In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In various embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is non-small cell lung cancer, and in some embodiments, is locally-advanced or metastatic non-small cell lung cancer.

In various embodiments, the patient exhibits at least a stable disease (SD), as measured by RECIST 1.1 protocol, after the combination period lasts 3, 6, or 8 months. In various embodiments, the patient exhibits at least a partial response (PR), as measured by RECIST 1.1 protocol, after the combination period lasts 3, 6, or 8 months. In various embodiments, the patient exhibits a progression free survival (PFS) of at least 3 months. In various embodiments, the patient exhibits fewer grade 3 or 4 treatment related adverse events (TRAEs) compared to a patient administered sotorasib and the anti-PD1 antibody or anti-PD-L1 antibody without an induction period.

In various embodiments, the patient has received at least one prior line of therapy. In various embodiments, the patient has not previously received treatment with an anti-PD1 or anti-PD-L1 immunotherapy. In various embodiments, the patient has previously received treatment with anti-PD1 or anti-PD-L1 immunotherapy. In various embodiments, the patient has previously received treatment with (i) anti-PD1 or anti-PDL1 immunotherapy or (ii) prior platinum-based combination chemotherapy. In various embodiments, the patient has previous received treatment with (i) anti-PD1 or anti-PD-L1 immunotherapy and (ii) prior platinum-based combination chemotherapy. In various embodiments, the patient has previously undergone an EGFR, ALK or ROS1 targeted therapy if the cancer also exhibited a mutation in EGFR, ALK, or ROS1. In various embodiments, the patient has progressed on an EGFR, ALK or ROS1 targeted therapy if the cancer also exhibited a mutation in EGFR, ALK, or ROS1. In various embodiments, the patient completed neoadjuvant or adjuvant chemotherapy at least 12 months prior to diagnosis of advanced stage cancer. In various embodiments, (1) (a) the patient has previously received treatment with (i) anti-PD1 or anti-PD-L1 immunotherapy or (ii) prior platinum-based combination chemotherapy; or (b) the patient has previously received treatment with (i) anti-PD1 or anti-PD-L1 therapy and (ii) prior platinum-based chemotherapy; and (2) the patient optionally has previously undergone an EGFR, ALK or ROS1 targeted therapy if the cancer also exhibited a mutation in EGFR, ALK, or ROS1. In various embodiments, the patient (1) has a cancer that exhibits a PD-L1 tumor proportion score (TPS) of 50% or greater; and (2) has not received any systemic therapy for locally advanced or metastatic non-small cell lung cancer; (i) but for a EGFR, ALK, or ROS1 targeted cancer therapy, if cancer exhibited a mutation in EGFR, ALK, or ROS1, and the patient has progressed on the targeted cancer therapy; and (ii) but for neoadjuvant or adjuvant chemotherapy completed at least 12 months prior to the start of the induction period and has not received immune checkpoint inhibitor therapy. In various embodiments, the patient (1) has a cancer that exhibits a PD-L1 tumor proportion score (TPS) of 1% or greater; and (2) has not received any systemic therapy for locally advanced or metastatic non-small cell lung cancer; (i) but for a EGFR, ALK, or ROS1 targeted cancer therapy, if cancer exhibited a mutation in EGFR, ALK, or ROS1, and the patient has progressed on the targeted cancer therapy; and (ii) but for neoadjuvant or adjuvant chemotherapy completed at least 12 months prior to the start of the induction period and has not received immune checkpoint inhibitor therapy.

### BRIEF DESCRIPTION OF THE FIGURES

FIG. 1 shows the efficacy of patients treated with a combination therapy of sotorasib with atezolizumab and of sotorasib with pembrolizumab, with or without a lead-in treatment of sotorasib alone, where CR is complete response, DCR is disease control rate, DpR is median depth of response, ORR is objective response rate, PD is progressive disease, PR is partial response, and SD is stable disease.

FIG. 2 shows the efficacy of sotorasib and pembrolizumab at different doses of sotorasib, with or without a lead-in sotorasib monotherapy, where ORR is objective response rate, and SD is stable disease.

### DETAILED DESCRIPTION

Kristen rat sarcoma (KRAS) G12C mutation has been identified as a putative oncogenic driver in several types of solid tumors including non-small cell lung cancer (NSCLC). Sotorasib, a covalent inhibitor of KRASG12C has shown promising anti-tumor activity in a Phase 1/2 Study (Clinical Trial 20170543). Inhibition of programmed death-ligand 1 (PD-L1) has been an effective strategy in the treatment of NSCLC. Preclinically, combined treatment with sotorasib and anti-programmed cell death protein-1 (PD-1) therapy increased infiltration of CD8+ T cells into tumors and greatly enhanced anti-tumor efficacy compared to treatment with either single agent in vivo (Canon et al, 2019). The addition of anti PD1 or PD-L1 therapy may therefore enhance the anti-tumor activity of therapy targeted to mutant KRAS.

The combination of immunotherapy agents (e.g., anti-PD1, anti-PDL-1, anti CTLA-4 therapy) and targeted small molecule therapies (e.g., epidermal growth factor receptor [EGFR] inhibitors, BRAF inhibitors, vascular endothelial growth factor [VEGF] inhibitors) has frequently led to an unexpected increase in frequency and/or severity of adverse events. Some examples include liver enzyme elevations with or without concomitant bilirubin elevation (ipilimumab and vemurafenib, durvalumab and gefitinib, pembrolizumab and axitinib, durvalumab and osimertinib, pembrolizumab and gefitinib) (Ahn et al, 2016; Gibbons et al, 2016; Ribas et al, 2013; Rini et al, 2019; Yang et al, 2019) and interstitial lung disease (osimertinib and durvalumab) (Ahn et al, 2016). Some approaches that have been explored to improve the tolerability of these combinations include modulating the dose of the targeted therapy agent and/or exploring sequential dosing with an initial run in for a period of 3 to 6 weeks with the targeted therapy agent followed by the initiation of the immunotherapy agent (Ahn et al, 2016; Sullivan et al, 2019).

Sotorasib in combination with a fixed dose of pembrolizumab (200 mg Q3W starting from day 1) is being explored given concurrently in a cohort of patients with previously treated NSCLC in Study 20170543. While no dose-limiting toxicities (DLTs) were observed at a dose of 960 mg and 720 mg of sotorasib, all patients enrolled at these dose levels experienced grade 3 adverse events requiring treatment interruptions and in some cases, treatment discontinuation. The study continues to explore a lower dose of sotorasib at 360 mg daily (QD) in combination with pembrolizumab. But, further investigations are warranted for a dosing regimen for sotorasib and a PD1 or PDL1 inhibitor that could minimize adverse events and avoid or reduce dose interruptions or treatment discontinuation.

The term “patient” as used herein refers to a subject, such as a human, who is in need of treatment with one or more methods disclosed herein.

### KRAS Pathway and PD-L1

The KRAS G12C mutation is estimated to occur in approximately 13% of lung adenocarcinoma (The American Association for Cancer Research [AACR] Project GENIE Consortium, 2017; Biernacka et al, 2016; Neumann et al, 2009). Enhancement of anti-tumor immunity through inhibition of PD-(L)-1 has been effective in treatment of many malignancies. Immune checkpoint inhibition (ICI) is now the mainstay of first-line treatment of metastatic NSCLC without EGFR or anaplastic lymphoma kinase (ALK) genomic tumor aberrations, either as monotherapy or as combination therapy (with chemotherapy±anti-angiogenic therapy). Results of published studies are still limited concerning the association between KRAS mutations and the response to anti-PD1 or anti-PD-L1 antibodies. Recent exploratory analysis from the KEYNOTE-042 study (Herbst et al, 2019) comparing first line pembrolizumab versus chemotherapy for patients with PD-L1 positive (tumor proportion score [TPS]≥1%) advanced NSCLC showed that patients with KRAS mutation show higher PD-L1 TPS (median 60%) compared with those without (median 35%). For patients with KRAS G12C mutation, median progression-free survival (PFS) was 15 months on the pembrolizumab arm versus 6 months on chemotherapy (HR 0.27; 95% CI: 0.10-0.71). Similarly, median overall survival (OS) was longer for patients who received pembrolizumab (median NR versus chemotherapy [8 months, HR 0.28; 95% CI: 0.09-0.86]). A meta-analysis of 3 studies that compared second-line ICI (nivolumab Checkmate 057 Study and atezolizumab OAK and POPLAR studies) with docetaxel showed that in patients with KRAS mutation, the pooled HR for overall survival was 0.65 (95% CI: 0.44-0.97; p=0.03) (Lee et al, 2018).

Results from the IMMUNOTARGET registry that investigated the efficacy of ICI in tumors with oncogenic driver mutations showed that response rates were higher in KRAS mutated NSCLC compared to other mutations like ALK or EGFR) (Mazieres et al, 2019). This is consistent with other published reports (Borghaei and Brahmer, 2016). However, KRAS mutated NSCLC may also express higher levels of PD-1 and PD-L1 (Chen et al, 2017).

The methods disclosed herein comprise administering sotorasib alone during an “induction period” then administering sotorasib and an anti-PD1 or anti-PDL1 antibody as a combination therapy (in a “combination period”). The induction period, where the patient is administered sotorasib alone, is for 14 to 48 days, e.g., 14 to 42 days, 21 to 48 days, or 21 to 42 days. In some embodiments, the induction period is 21 days. In some embodiments, the induction period is 42 days. The combination period is the period of time the patient is on the combination therapy as disclosed in the methods herein. The combination period can be at least 30 days, at least 2 months, at least 3 months, at least 6 months, at least 8 months, at least 12 months, at least 18 months, at least 2 years, or at least 3 years. In various embodiments, the combination period ends when the patient demonstrates a complete response (CR), a partial response (PR), or stable disease (SD) as determined by Response Evaluation Criteria in Solid Tumors (RECIST) 1.1 protocol (Eisenhauer, et al., 2009).

For the methods disclosed herein, it has been discovered that administration of sotorasib to a patient for an induction period prior to administration of sotorasib with an anti-PD1 antibody or anti-PD-L1 antibody resulted in fewer grade 3 or 4 treatment related adverse events (TRAEs), compared to a patient administered sotorasib and the anti-PD1 antibody or anti-PD-L1 antibody without an induction period. Fewer severe TRAEs means a safer treatment protocol, and may allow the patient to stay on the treatment protocol longer, which may provide a more effective treatment for the cancer.

### Sotorasib

Sotorasib is a small molecule that irreversibly inhibits the KRASG12C mutant protein. Sotorasib is also referred to as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure:

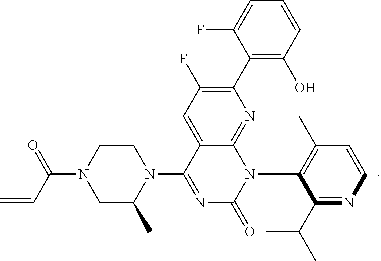

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision May 2021), which is herein incorporated by reference in its entirety.

### Anti-PD1 or Anti-PD-L1 Antibodies

The anti-PD1 antibody used in the methods disclosed herein can be, for example, balstilimab, budigalimab, cadonilimab, camrelizumab, cetrelimab, cemiplimab, dostarlimab, ezabenlimab, finotonlimab, nivolumab, penpulimab, pembrolizumab, pucotenlimab, retifanlimab, rulonilimab, sasanlimab, serplulimab, sintilimab, spartalizumab, tebotelimab, tislelizumab, toripalimab, zeluvalimab (AMG 404), or zimberelimab. In some embodiments, the anti-PD1 antibody is cemiplimab, dostarlimab, pembrolizumab, or nivolumab. In some embodiments, the anti-PD1 antibody is pembrolizumab (KEYTRUDA®). Pembrolizumab is a humanized immunoglobulin G4 monoclonal antibody (mAb) with high specificity of binding to the PD1 receptor, thus inhibiting its interaction with PD-L1 and PD-L2. Pembrolizumab is approved as a single agent for the first line treatment of patients with metastatic NSCLC expressing PD-L1 (TPS≥1%) as determined by an FDA-approved test, with disease progression on or after platinum containing chemotherapy. Patients with EGFR or ALK genomic tumor aberrations should have disease progression on FDA approved therapy for these aberrations prior to receiving pembrolizumab. Complete information about pembrolizumab (KEYTRUDA®) preparation, dispensing, dosage, and administration schedule can be found in the local package insert (for the United States see, e.g., KEYTRUDA® U.S. Prescribing Information, Merck & Co., Inc., Whitehouse Station, New Jersey, 08889 (revision February 2022), which is herein incorporated by reference in its entirety).

The anti-PD-L1 antibody used in the methods disclosed herein can be, for example, adebrelimab, atezolizumab, avelumab, cosibelimab, durvalumab, envafolimab, erfonrilimab, garivulimab, lodapolimab, opucolimab, sugemalimab, socazolimab, or tagitanlimab. In various embodiments, the anti-PD-L1 antibody is atezolizumab (TECENTRIQ®), durvalumab (IMFINZI®), or avelumab (BAVENCIO®). In various embodiments, the anti-PD-L1 antibody is atezolizumab (TECENTRIQ®). Complete information about atezolizumab (TECENTRIQ®) preparation, dispensing, dosage, and administration schedule can be found in the local package insert (for the United States see, e.g., TECENTRIQ® U.S. Prescribing Information, Genentech, Inc., South San Francisco, California, 94080 (revision January 2022), which is herein incorporated by reference in its entirety). Complete information about durvalumab (IMFINZI®) preparation, dispensing, dosage, and administration schedule can be found in the local package insert (for the United States see, e.g., IMFINZI® U.S. Prescribing Information, AstraZeneca Pharmaceuticals LP, Wilmington, Delaware, 19850 (revision July 2021), which is herein incorporated by reference in its entirety). Complete information about avelumab (BAVENCIO®) preparation, dispensing, dosage, and administration schedule can be found in the local package insert (for the United States see, e.g., BAVENCIO® U.S. Prescribing Information, EMD Serono, Inc., Rockland, Maryland, 02370 (revision 11/2020), which is herein incorporated by reference in its entirety).

### Dosing Regimens

The methods disclosed herein comprise administering a therapeutically effective amount of sotorasib (alone during the induction period) and a therapeutically effective amount of sotorasib and a therapeutically effective amount an anti-PD1 or anti-PD-L1 antibody as a combination therapy (during the combination period).

In some embodiments, the methods comprise administering 960 mg sotorasib to the patient during the induction period. In some embodiments, the methods comprise administering 360 mg sotorasib to the patient during the induction period. In some embodiments, the methods comprise administering 240 mg sotorasib to the patient during the induction period. In some embodiments, the methods comprise administering 120 mg sotorasib to the patient during the induction period. In some embodiments, the sotorasib is administered to the patient once daily during the induction period. In some embodiments, the sotorasib is administered to the patient twice daily during the induction period, wherein each dose of sotorasib corresponds to half of the therapeutically effective amount administered during the induction period.

In some embodiments, the methods comprise administering 960 mg sotorasib to the patient during the combination period. In some embodiments, the methods comprise administering 360 mg sotorasib to the patient during the combination period. In some embodiments, the methods comprise administering 240 mg sotorasib to the patient during the combination period. In some embodiments, the methods comprise administering 120 mg sotorasib to the patient during the combination period. In some embodiments, the sotorasib is administered to the patient once daily during the combination period. In some embodiments, the sotorasib is administered to the patient twice daily during the combination period, wherein each dose of sotorasib corresponds to half of the therapeutically effective amount administered during the combination period.

In some embodiments, the anti-PD-L1 antibody administered to the patient during the combination period in the methods disclosed herein is atezolizumab, avelumab, or durvalumab. In some embodiments, the anti-PD-L1 antibody administered during the combination period is atezolizumab. In various embodiments, the anti-PD1 antibody administered to the patient during the combination period in the methods disclosed herein is cemiplimab, dostarlimab, pembrolizumab, or nivolumab. In some embodiments, the anti-PD1 antibody administered during the combination period is pembrolizumab. In some embodiments, pembrolizumab is administered during the combination period at a therapeutically effective amount of 200 mg. In some embodiments, the 200 mg pembrolizumab is administered via IV to the patient once every three weeks during the combination period.

In some embodiments, the patient is administered 360 mg sotorasib orally once per day during the induction period and during the combination period and 200 mg pembrolizumab via IV once every three weeks during the combination period.

In some embodiments, the patient is administered 960 mg sotorasib orally once per day during the induction period and during the combination period and 200 mg pembrolizumab via IV once every three weeks during the combination period.

In some embodiments, the patient is administered 240 mg sotorasib orally once per day during the induction period and during the combination period and 200 mg pembrolizumab via IV once every three weeks during the combination period.

In some embodiments, the patient is administered 120 mg sotorasib orally once per day during the induction period and during the combination period and 200 mg pembrolizumab via IV once every three weeks during the combination period.

In various embodiments, sotorasib is administered with food. In various embodiments, sotorasib is administered without food.

In various embodiments, the patient is in further need of treatment with an acid-reducing agent. Acid-reducing agents include, but are not limited to, a proton pump inhibitor (PPI), a H2 receptor antagonist (H2RA), and a locally acting antacid. In some embodiments, the patient is further in need of treatment with a PPI or a H2RA. Exemplary PPIs include, but are not limited to, omeprazole, pantoprazole, esomeprazole, lansoprazole, rabeprazole, or dexlansoprazole. Exemplary H2RAs include, but are not limited to, famotidine, ranitidine, cimetidine, nizatidine, roxatidine and lafutidine. Exemplary locally acting antacids include, but are not limited to, sodium bicarbonate, calcium carbonate, aluminum hydroxide, and magnesium hydroxide. In some embodiments, the patient, who is in further need of treatment with an acid reducing agent, is not administered a proton pump inhibitor or a H2 receptor antagonist in combination with sotorasib. In some embodiments, the patient, who is in further need of treatment with an acid-reducing agent, is not administered a proton pump inhibitor or a H2 receptor antagonist in combination with sotorasib, but is administered a locally acting antacid in combination with sotorasib. In some embodiments, sotorasib is administered about 4 hours before or about 10 hours after a locally acting antacid.

In various embodiments, the patient is in further need of treatment with a CYP3A4 inducer. In some embodiments, the patient is not administered a CYP3A4 inducer in combination with sotorasib. Exemplary CYP3A4 inducers include, but are not limited to, apalutamide, avasimibe, barbiturate, brigatinib, carbamazepine, clobazam, dabrafenib, efavirenz, elagolix, enzalutamide, eslicarbazepine, glucocorticoids, ivosidenib, letermovir, lorlatinib, lumacaftor, mitotane, modafinil, nevirapine, oritavancin, oxcarbazepine, perampanel, phenobarbital, phenytoin, pioglitazone, rifabutin, rifampin, rifapentine, St. John's wort, telotristat, and troglitazone. See, e.g., Flockhart D A, Drug Interactions: Cytochrome P450 Drug Interaction Table. Indiana University School of Medicine (2007), www.drug-interactions.medicine.iu.edu, accessed May 2021. In some embodiments, the patient is not administered a strong CYP3A4 inducer in combination with sotorasib. Exemplary strong CYP3A4 inducers include, but are not limited to, rifampin, mitotane, avasimibe, rifapentine, apalutamide, ivosidenib, phenytoin, carbamazepine, enzalutamide, St John's Wort extract, and lumacaftor. See, e.g., www.fda.gov/drugs/drug-interactions-labeling/drug-development-and-drug-interactions-table-substrates-inhibitors-and-inducers, accessed May 2021.

In various embodiments, the patient is in further need of treatment with a CYP3A4 substrate. In some embodiments, the patient is not administered a CYP3A4 substrate in combination with sotorasib. Exemplary CYP3A4 substrates include, but are not limited to, abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus (fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone. See, e.g., Flockhart D A, Drug Interactions: Cytochrome P450 Drug Interaction Table. Indiana University School of Medicine (2007), https://drug-interactions.medicine.iu.edu, accessed May 2021. In some embodiments, the CYP3A4 substrate is a CYP3A4 substrate with a narrow therapeutic index. Exemplary CYP3A4 substrates with a narrow therapeutic index include, but are not limited to, alfentanil, cyclosporine, dihydroergotamine, ergotamine, everolimus, fentanyl, primozide, quinidine, tacrolimus, and sirolimus.

In various embodiments, the patient is in further need of treatment with a P-glycoprotein (P-gp) substrate. In some embodiments, the patient is not administered a P-gp substrate in combination with sotorasib. Exemplary P-gp substrates include, but are not limited to, etexilate, digoxin, fexofenadine, everolimus, cyclosporine, sirolimus, tacrolimus, and vincristine. See, e.g., www.fda.gov/drugs/drug-interactions-labeling/drug-development-and-drug-interactions-table-substrates-inhibitors-and-inducers, accessed May 2021. In some embodiments, the patient is not administered a P-gp substrate in combination with sotorasib, wherein the P-gp substrate is a P-gp substrate with a narrow therapeutic index. Exemplary P-gp substrates with a narrow therapeutic index include, but are not limited to, digoxin, everolimus, cyclosporine, tacrolimus, sirolimus, and vincristine.

### Determination of KRAS G12C Mutation in Cancer

The patient treated in the methods disclosed herein is one suffering from a cancer having a KRAS G12C mutation. In various embodiments, the patient has a cancer that was determined to have one or more cells expressing the KRAS^{G12C} mutant protein prior to administration of sotorasib as disclosed herein. The presence or absence of G12C mutation in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences of wild-type human KRAS (nucleotide sequence set forth in Genbank Accession No. BC010502; amino acid sequence set forth in Genbank Accession No. AGC09594) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; Alizadeh et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is a fresh tumor/cancer sample. In some embodiments, the sample is a frozen tumor/cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

### KRAS G12C Mutated Cancers

The methods described herein comprise treating a cancer with a KRAS G12C mutation in a patient. Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020).

Sotorasib was evaluated in a Phase 1 dose escalation and expansion trial with 129 patients having histologically confirmed, locally advanced or metastatic cancer with the KRAS G12C mutation identified by local molecular testing on tumor tissues, including 59 patients with non-small cell lung cancer, 42 patients with colorectal cancer, and 28 patients with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6).

KRAS G12C mutations occur with the alteration frequencies shown in the table below (Cerami et al., 2012; Gao et al., 2013). For example, the table shows that 11.6% of patients with non-small cell lung cancer have a cancer, wherein one or more cells express KRAS G12C mutant protein. Accordingly, sotorasib, which specifically and irreversibly bind to KRASG12C is useful for treatment of patients having a cancer, including, but not limited to the cancers listed in Table 1 below.

TABLE 1

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine Tumor | 0.4 |
| Bladder Cancer | 0.4 |
| Myelodysplastic/Myeloproliferative Neoplasms | 0.3 |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In some embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is non-small cell lung cancer, and in some specific embodiments, metastatic or locally advanced non-small cell lung cancer. In various embodiments, the cancer is colorectal cancer. In some embodiments, the cancer is pancreatic cancer.

### Methods of Detecting PD-L1 Protein Expression

PD-L1 expression can be determined by methods known in the art. For example, PD-L1 expression can be detected using PD-L1 IHC 22C3 pharmDx, an FDA-approved in vitro diagnostic immunohistochemistry (IHC) test developed by Dako and Merck as a companion test for treatment with pembrolizumab. This is qualitative assay using Monoclonal Mouse Anti-PD-L1, Clone 22C3 PD-L1 and EnVision FLEX visualization system on Autostainer Lin 48 to detect PD-L1 in FFPE samples, such as human non-small cell lung cancer tissue. Expression levels can be measured using the tumor proportion score (TPS), which measures the percentage of viable tumor cells showing partial or complete membrane staining at any intensity. Staining can show PD-L1 expression from 0% to 100%.

PD-L1 expression can also be detected using PD-L1 IHC 28-8 pharmDx, the FDA-approved in vitro diagnostic immunohistochemistry (IHC) test developed by Dako and Bristol-Myers Squibb as a companion test for treatment with nivolumab. This qualitative assay uses the Monoclonal rabbit anti-PD-L1, Clone 28-8 and EnVision FLEX visualization system on Autostainer Lin 48 to detect PD-L1 in formalin-fixed, paraffin-embedded (FFPE) human cancer tissue.

Other commercially available tests for PD-L1 detection include the Ventana SP263 assay (developed by Ventana in collaboration with AstraZeneca) that utilizes monoclonal rabbit anti-PD-L1, Clone SP263 and the Ventana SP142 Assay (developed by Ventana in collaboration with Genentech/Roche) that uses rabbit monoclonal anti-PD-L1 clone SP142.

In some embodiments, a test approved by a regulatory authority, such as the US Food and Drug Administration (FDA), is used to determine the PD-L1 TPS of a cancer as disclosed herein. In various embodiment, the PD-L1 TPS is determined using a immunohistochemistry (IHC) test. In some embodiments, the IHC test is the PD-L1 IHC 22C3 pharmDx test. In various embodiments, the IHC test conducted with samples acquired by, for example, resection, CNB, or FNA.

In various embodiment, the patient has a PD-L1 TPS of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than 50%, or less than 1%. In various embodiments, the patient has a PD-L1 TPS of more than or equal to 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 50%, or less than or equal to 1%. In various embodiments, the patient has a PD-L1 TPS of more than 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS score a range bound by any of the values cited in the foregoing embodiments. For example, the patient has a PD-L1 TPS score in the range of less than 50% and more than or equal to 1%, less than or equal to 50% and more than 1%, less than or equal to 50% and more than or equal to 1%, or less than 50% and more than 1%.

In various embodiments, the patient has a PD-L1 TPS score in the range of 1% to 49%. In some embodiments, the patient has a PD-L1 TPS score in the range of more than or equal to 0% and less than 1%. In some embodiments, the patient has a PD-L1 TPS score in the range of 50% or more and less than or equal to 100%. In some embodiments, the patient has a PD-L1 TPS score of less than 1%. In some embodiments, the patient has a PD-L1 TPS score of 1% or more. In some embodiments, the patient as a PD-L1 TPS score of 1-49%. In some embodiments, the patient has a PD-L1 TPS score of 50% or more (i.e., 50% to 100%).

### Prior Cancer Therapies of Patient Treated in Disclosed Methods

In some embodiments, the patient is a treatment-naïve patient—i.e., the patient has not received any prior treatment for the cancer having a KRAS G12C mutation being treated in the methods disclosed herein.

In some embodiments, the patient treated in the methods described herein has been previously treated with a different anti-cancer therapy, e.g., at least one-such as one, or two, or three-other systemic cancer therapy. In some embodiments, the patient had previously been treated with one other systemic cancer therapy, such that the therapy described herein is a second line therapy. In some embodiments, the patient had previously been treated with two other systemic cancer therapy, such that the therapy as provided herein is a third line therapy.

In some embodiments, the prior systemic cancer therapy is not a therapy with a KRAS^{G12C} inhibitor. In some embodiments, the KRAS^{G12C} inhibitor is sotorasib, adagrasib, GDC-6036, D-1553, JDQ443, LY3484356, BI1823911, JAB-21822, RMC-6291, or APG-1842. In certain embodiments, the KRAS G12C inhibitor is sotorasib. In certain embodiments, the KRAS^{G12C} inhibitor is adagrasib. In some embodiments, the therapy is monotherapy. In one embodiment, the therapy with a KRAS^{G12C} inhibitor is sotorasib monotherapy. In another embodiment, the therapy with a KRAS^{G12C} inhibitor is monotherapy with adagrasib.

Prior systemic cancer therapies include, but are not limited to, chemotherapies and immunotherapies. Specific contemplated prior systemic cancer therapies include, but are not limited to, anti-PD1 immunotherapy, anti-PD-L1 immunotherapy, and platinum based chemotherapy. Some examples of anti-PD1 immunotherapy and anti-PD-L1 immunotherapy include, but are not limited to, pembrolizumab, nivolumab, cemiplimab, tisielizumab, toripalimab, aspartalizumab, dostarlimab, retifanlimab, simtilimab, pidilizumab atezolizumab, avelumab, durvalumab, and zeluvalimab (AMG 404). Some examples of platinum based chemotherapies include, but are not limited to, carboplatin, oxaliplatin, cisplatin, nedaplatin, satraplatin, lobaplatin, triplatin tetranitrate, picoplatin, ProLindac, and aroplatin. In some embodiments, the platinum based chemotherapy is in combination with a second chemotherapeutic (e.g., a platinum based combination chemotherapy), and such second chemotherapeutics can be, for example, paclitaxel, docetaxel, gemcitabine, or pemetrexed.

In some embodiments, the patient has previously been administered a systemic cancer therapy that is a targeted therapy if the cancer was identified to have an actionable oncogenic driver mutation in the epidermal growth factor receptor gene (EGFR), anaplastic lymphoma kinase gene (ALK), and/or ROS proto-oncogene 1 (ROS1). In some embodiments, the patient progressed on an EGFR, ALK, or ROS1 targeted therapy. Targeted therapies for EGFR mutations include, but are not limited to, cetuximab, panitumumab, erlotinib, gefitinib, and afatinib. Targeted therapies for ALK mutations include, but are not limited to, crizotinib, entrectinib, lorlatinib, repotrectinib, brigatinib, alkotinib, alectinib, ensartinib, and ceritinib. Targeted therapies for ROS1 mutations include, but are not limited to, crizotinib, entrecetinib, ensartinib, alkotinib, brigatinib, taletrectinib, cabozantinib, repotrectinib, lorlatinib, and ceritinib.

In some embodiments, (1) (a) the patient has previously received treatment with (i) anti-PD1 or anti-PD-L1 immunotherapy or (ii) prior platinum-based combination chemotherapy; or (b) the patient has previously received treatment with (i) anti-PD1 or anti-PD-L1 therapy and (ii) prior platinum-based chemotherapy; and (2) the patient optionally has previously undergone an EGFR, ALK or ROS1 targeted therapy if the cancer also exhibited a mutation in EGFR, ALK, or ROS1.

In some embodiments, the patient (1) has a cancer that exhibits a PD-L1 tumor proportion score (TPS) of 50% or greater; and (2) has not received any systemic therapy for locally advanced or metastatic non-small cell lung cancer; (i) but for a EGFR, ALK, or ROS1 targeted cancer therapy, if cancer exhibited a mutation in EGFR, ALK, or ROS1, and the patient has progressed on the targeted cancer therapy; and (ii) but for neoadjuvant or adjuvant chemotherapy completed at least 12 months prior to the start of the induction period and has not received immune checkpoint inhibitor therapy. In various embodiments, the patient (1) has a cancer that exhibits a PD-L1 tumor proportion score (TPS) of 1% or greater; and (2) has not received any systemic therapy for locally advanced or metastatic non-small cell lung cancer; (i) but for a EGFR, ALK, or ROS1 targeted cancer therapy, if cancer exhibited a mutation in EGFR, ALK, or ROS1, and the patient has progressed on the targeted cancer therapy; and (ii) but for neoadjuvant or adjuvant chemotherapy completed at least 12 months prior to the start of the induction period and has not received immune checkpoint inhibitor therapy.

In various embodiments, the patient exhibits an Eastern Cooperative Oncology Group (ECOG) performance status of 0, 1 or 2 (see, e.g., Zubrod et al., 1960). Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare and totally confined to bed or chair. Status 5 indicates death.

### Response to Sotorasib and Pembrolizumab Therapy As Disclosed Herein

Response rates or results for patients administered the therapy (i.e., sotorasib and pembrolizumab) in the methods disclosed herein can be measured in a number of ways, after the patient has been taking the therapy (in the combination period) for a suitable length of time. In various embodiments, a patient is administered the therapy (in the combination period) for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months (in the combination period). In various embodiments, the patient is administered the therapy for at least 1 month (in the combination period). In various embodiments, the patient is administered the therapy for at least 3 months (in the combination period). In various embodiments, the patient is administered the therapy for at least 6 months (in the combination period). In various embodiments, the patient is administered the therapy for at least 8 months (in the combination period).

The patient can respond to the therapy as measured by at least a stable disease (SD), as determined by Response Evaluation Criteria in Solid Tumors (RECIST) 1.1 protocol (Eisenhauer, et al., 2009). An at least stable disease is one that is a stable disease, has shown a partial response (PR) or has shown a complete response (CR) (i.e., “at least SD”=SD+PR+CR, often referred to as disease control). In various embodiments, the stable disease is neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Progression free survival (PFS) can be assessed as described in the RECIST 1.1 protocol. In various embodiments, the patient exhibits a PFS of at least 1 month. In various embodiments, the patient exhibits a PFS of at least 3 months. In some embodiments, the patient exhibits a PFS of at least 6 months.

Additional means for assessing response are described in detail in the examples below and can generally be applied to the methods disclosed herein.

### Adverse Events

The term “adverse event” or “(AE)” as used herein refers to any unfavorable and unintended sign (including an abnormal laboratory finding), symptom, or disease temporally associated with the use of a medical treatment or procedure that may be considered related to the medical treatment or procedure.

In some embodiments, the adverse event is hepatotoxicity (e.g., elevation of liver enzymes), interstitial lung disease (ILD)/pneumonitis, diarrhea, and/or nausea/vomiting.

### Hepatotoxicity

In some embodiments, the adverse event is hepatotoxicity. The term “hepatotoxicity” as used herein refers to a patient having abnormal laboratory values of liver biomarkers (e.g., alkaline phosphatase (ALP), aspartate amino transferase (AST), alanine aminotransferase (ALT), and/or total bilirubin (TBL)), when the patient had baseline levels of the liver biomarker(s) prior to sotorasib administration that were not abnormal laboratory values or were lower than those measured after administration of sotorasib.

Alanine transaminase (ALT), also called serum glutamic pyruvate transaminase (SGPT) or alanine aminotransferase (ALAT), catalyzes the transfer of an amino group from alanine to a-ketoglutarate to produce pyruvate and glutamate. When the liver is damaged, levels of ALT in the blood can rise due to the leaking of ALT into the blood from damaged or necrosed hepatocytes.

Aspartate transaminase (AST) also called serum glutamic oxaloacetic transaminase (SGOT or GOT) or aspartate aminotransferase (ASAT), catalyzes the transfer of an amino group from aspartate to a-ketoglutarate to produce oxaloacetate and glutamate. AST can increase in response to liver damage. Elevated AST also can result from damage to other sources, including red blood cells, cardiac muscle, skeletal muscle, kidney tissue, and brain tissue. The ratio of AST to ALT can be used as a biomarker of liver damage.

Bilirubin is a catabolite of heme that is cleared from the body by the liver. Conjugation of bilirubin to glucuronic acid by hepatocytes produces direct bilirubin, a water-soluble product that is readily cleared from the body. Indirect bilirubin is unconjugated, and the sum of direct and indirect bilirubin constitutes total bilirubin. Elevated total bilirubin can be indicative of liver impairment.

Alkaline phosphatase (ALP) hydrolyzes phosphate groups from various molecules and is present in the cells lining the biliary ducts of the liver. ALP levels in plasma can rise in response to liver damage and are higher in growing children and elderly patients with Paget's disease. However, elevated ALP levels usually reflect biliary tree disease.

In some embodiments, the patient is not suffering from a disorder that results in elevated liver biomarkers. Disorders associated with elevated liver biomarkers (such as AST/ALT and/or TBL values) include, but are not limited to, hepatobiliary tract disease; viral hepatitis (e.g., hepatitis A/B/C/D/E, Epstein-Barr Virus, cytomegalovirus, herpes simplex virus, varicella, toxoplasmosis, and parvovirus); right sided heart failure, hypotension or any cause of hypoxia to the liver causing ischemia; exposure to hepatotoxic agents/drugs or hepatotoxins, including herbal and dietary supplements, plants and mushrooms; heritable disorders causing impaired glucuronidation (e.g., Gilbert's syndrome, Crigler-Najjar syndrome) and drugs that inhibit bilirubin glucuronidation (e.g., indinavir, atazanavir); alpha-one antitrypsin deficiency; alcoholic hepatitis; autoimmune hepatitis; Wilson's disease and hemochromatosis; nonalcoholic fatty liver disease including steatohepatitis; and/or non-hepatic causes (e.g., rhabdomyolysis, hemolysis).

Prior to receiving sotorasib, the baseline liver function of the patient can be assessed by various means known in the art, such as blood chemistry tests measuring biomarkers of liver function. In some embodiments, the methods described herein comprise monitoring liver biomarkers in the patient and withholding sotorasib administration in patients having >Grade 2 abnormal liver function, as assessed by levels of AST and/or ALT. In such embodiments, sotorasib administration is paused until the AST and/or ALT levels in the patient improve(s) to Grade 1 or better (baseline).

Adverse effect Grades for abnormal liver function are defined herein by the modified Common Toxicity Criteria (CTC) provided in Table 2. See the National Cancer Institute Common Terminology Criteria for Adverse Events v5.0 (NCI CTCAE) published Nov. 27, 2017 by the National Cancer Institute, incorporated herein by reference in its entirety.

TABLE 2
Common Toxicity Criteria
Toxicity grades

| Toxicity | 0 | 1 | 2 | 3 | 4 |
|---|---|---|---|---|---|
| ALT | WNL | >ULN-3.0 × ULN, | >3-5 × ULN, | >5-20 × ULN, | >20 × ULN if |
| | | if baseline was | if baseline was | if baseline was | baseline was |
| | | normal; 1.5-3.0 × | normal, >3.0- | normal; >5.0- | normal; >20 × |
| | | baseline if baseline | 5.0 × baseline | 20.0 × baseline | baseline if |
| | | was abnormal | if baseline was | if baseline was | baseline was |
| | | | abnormal | abnormal | abnormal |
| AST | WNL | >ULN-3.0 × ULN | >3-5 × ULN | >5-20 × ULN | >20 × ULN if |
| | | if baseline was | if baseline was | if baseline was | baseline was |
| | | normal; 1.5-3.0 × | normal, >3.0- | normal; >5.0- | normal; >20 × |
| | | baseline if baseline | 5.0 × baseline | 20.0 × baseline | baseline if |
| | | was abnormal | id baseline was | if baseline was | baseline was |
| | | | abnormal | abnormal | abnormal |
| Bilirubin | WNL | >ULN-1.5 × ULN | >1.5-3 × ULN | >3-10 × ULN | >10 × ULN if |
| | | if baseline was | if baseline was | if baseline was | baseline was |
| | | normal; >1.0-1.5 × | normal; >1.5- | normal; >3.0- | normal; >10.0 × |
| | | baseline if baseline | 3.0 × baseline | 10 × baseline | baseline if |
| | | was abnormal | if baseline was | if baseline was | baseline was |
| | | | abnormal | abnormal | abnormal |
| ALP | WNL | >ULN-2.5 × ULN | >2.5-5.0 × ULN | >5-20 × ULN | >20 × ULN if |
| | | if baseline was | if baseline was | if baseline was | baseline was |
| | | normal; 2.0-2.5 × | normal, >2.5- | normal; >5.0- | normal; >20 × |
| | | baseline if baseline | 5.0 × baseline | 20.0 × baseline | baseline if |
| | | was abnormal | if baseline was | if baseline was | baseline was |
| | | | abnormal | abnormal | abnormal |

ALP = alkaline phosphatase;
ALT = alanine aminotransferase;
AST = aspartate aminotransferase;
ULN = upper limit of normal;
WNL = within normal limits

Grade 0 levels are characterized by biomarker levels within normal limits (WNL). “Normal” liver function, as used herein, refers to Grade 0 adverse effects. “Abnormal” liver function, as used herein, refers to Grade 1 and above adverse effects.

“Grade 1 liver function abnormalities” include elevations in ALT or AST greater than the ULN and less than or equal to 3-times the ULN if baseline was normal; 1.5-3.0× baseline if baseline was abnormal. Grade 1 liver function abnormalities also include elevations of bilirubin levels greater than the ULN and less than or equal to 1.5-times the ULN if baseline was normal; >1.0-1.5× baseline if baseline was abnormal. Grade 1 liver function abnormalities also include elevations of ALP greater than the ULN and less than or equal to 2.5-times the ULN if baseline was normal; >2.0-2.5× baseline if baseline was abnormal.

“Grade 2 liver function abnormalities” include elevations in ALT or AST greater than 3-times and less than or equal to 5-times the upper limit of normal (ULN) if baseline was normal; >3.0-5.0× baseline if baseline was abnormal. Grade 2 liver function abnormalities also include elevations of bilirubin levels greater than 1.5-times and less than or equal to 3-times the ULN if baseline was normal; >1.5-3.0× baseline if baseline was abnormal. Grade 2 liver function abnormalities also include elevations of ALP greater than 2.5-times and less than or equal to 5-times the ULN if baseline was normal; >2.5-5.0× baseline if baseline was abnormal.

“Grade 3 liver function abnormalities” include elevations in ALT, AST, or ALP greater than 5-times and less than or equal to 20-times the ULN if baseline was normal; >5.0-20.0× baseline if baseline was abnormal. Grade 3 liver function abnormalities also include elevations of bilirubin levels greater than 3-times and less than or equal to 10-times the ULN if baseline was normal; >3.0-10× baseline if baseline was abnormal.

“Grade 4 liver function abnormalities” include elevations in ALT, AST, or ALP greater than 20-times the ULN if baseline was normal; >20× baseline if baseline was abnormal. Grade 4 liver function abnormalities also include elevations of bilirubin levels greater than 10 times the ULN if baseline was normal; >10.0× baseline if baseline was abnormal.

The ULN for various indicators of liver function depends on the assay used, the patient population, and each laboratory's normal range of values for the specified biomarker, but can readily be determined by the skilled practitioner. Exemplary values for normal ranges for a healthy adult population are set forth in Table 3 below. See Cecil Textbook of Medicine, pp. 2317-2341, W.B. Saunders & Co. (1985).

TABLE 3
UPPER LIMIT OF NORMAL (ULN) VALUES

| | | |
|---|---|---|
| ALT | 8-20 | U/L |
| AST | 8-20 | u/l |
| Bilirubin | 0.2-1 | mg/dL |
| | 3.4-17.1 | μmol/L |
| ALP | 20-70 | U/L |

In any of the methods described herein, the total daily dose of sotorasib is reduced (e.g., from 960 mg to 480 mg, or from 480 mg to 240 mg) when the AST and/or ALT level(s) in the patient is/are elevated, e.g., to a Grade 2 or Grade 3 level, where the baseline AST and/or ALT levels of the patient were below Grade 2 or Grade 3 levels. In some embodiments, the total daily dose of sotorasib is reduced (e.g., from 960 mg to 480 mg, or from 480 mg to 240 mg), when the AST and/or ALT level(s) in the patient is/are elevated is to a Grade 1 level, wherein the baseline AST and/or ALT levels of the patient were below Grade 1 levels.

Alternatively, in any of the methods disclosed herein, the total daily dose of sotorasib is reduced (e.g., from 960 mg to 480 mg, or from 480 mg to 240 mg) when (1) AST and bilirubin levels in the patient are elevated, or (2) when AST or ALP levels in the patient are elevated, or (3) when ALT and bilirubin levels in the patient are elevated, or (4) when ALT and ALP levels in the patient are elevated, or (5) when bilirubin and ALP levels in the patient are elevated, e.g., to a Grade 1, Grade 2, Grade 3 or Grade 4 level, wherein the baseline AST, bilirubin, ALP, and/or ALT levels of the patient were below Grade 1, Grade 2, Grade 3 or Grade 4 levels, respectively. Alternatively, in any of the methods disclosed herein, three biomarkers of liver function may be elevated in the patient (e.g., ALT and AST and bilirubin, or ALT and AST and ALP) to a Grade 1, Grade 2, Grade 3 or Grade 4 level, wherein the baseline biomarker levels of the patient were below Grade 1, Grade 2, Grade 3 or Grade 4 levels, respectively.

In some embodiments, the total daily dose of sotorasib is reduced (e.g., from 960 mg to 480 mg, or from 480 mg to 240 mg) when the level of ALT and/or AST is greater than about 3 times compared to the upper limit of normal (ULN). In a related embodiment, the abnormal level of ALT and/or AST is greater than about 3-to about 5-fold increase compared to the upper limit of normal (ULN), i.e., a “Grade 2 abnormality”. In some embodiments, where the patient has an abnormal baseline, the Grade 2 abnormality is an abnormal level of ALT and/or AST greater than about 3-fold to about 5-fold increase compared to baseline. In some embodiments, the abnormal level of ALP is greater than about 2.5- to about 5-fold increase compared to the upper limit of normal (ULN), i.e., a “Grade 2 abnormality”. In some embodiments, where the patient has an abnormal baseline, the Grade 2 abnormality is an abnormal level of ALP greater than about 2.5-fold to about 5-fold increase compared to baseline. In some embodiments, the abnormal level of bilirubin is greater than about 1.5- to about 3-fold increase compared to the upper limit of normal (ULN), i.e., a “Grade 2 abnormality”. In some embodiments, where the patient has an abnormal baseline, the Grade 2 abnormality is an abnormal level of bilirubin greater than about 1.5-fold to about 3-fold increase compared to baseline.

In some embodiments, the total daily dose of sotorasib is reduced (e.g., from 960 mg to 480 mg, or from 480 mg to 240 mg) when the level of ALT and/or AST is greater than about 5 times compared to the upper limit of normal (ULN). In some embodiments, the total daily dose is reduced when the level of ALT, AST, or ALP is greater than about 5-to about 20-fold increase compared to the upper limit of normal (ULN), i.e., a “Grade 3 abnormality”. In some embodiments, where the patient has an abnormal baseline, the Grade 3 abnormality is an abnormal level of ALT and/or AST greater than about 5-fold to about 20-fold increase compared to baseline. In some embodiments, the abnormal level of ALP is greater than about 5-to about 20-fold increase compared to the upper limit of normal (ULN), i.e., a “Grade 3 abnormality”. In some embodiments, where the patient has an abnormal baseline, the Grade 3 abnormality is an abnormal level of ALP greater than about 5-fold to about 20-fold increase compared to baseline. In some embodiments, the total daily dose is reduced when the level of bilirubin is greater than about 3-to about 10-fold increase compared to the upper limit of normal (ULN), i.e., a “Grade 3 abnormality”. In some embodiments, where the patient has an abnormal baseline, the Grade 3 abnormality is an abnormal level of bilirubin greater than about 3-fold to about 10-fold increase compared to baseline.

In some embodiments, the total daily dose of sotorasib is reduced (e.g., from 960 mg to 480 mg, or from 480 mg to 240 mg) when the level of ALT and/or AST is greater than about 20 times compared to the upper limit of normal (ULN) (i.e., a “Grade 4 abnormality”). In some embodiments, where the patient has an abnormal baseline, the Grade 4 abnormality is an abnormal level of ALT and/or AST greater than about 20-fold increase compared to baseline. In some embodiments, the abnormal level of ALP is greater than about 20-fold increase compared to the upper limit of normal (ULN), i.e., a “Grade 4 abnormality”. In some embodiments, where the patient has an abnormal baseline, the Grade 4 abnormality is an abnormal level of ALP greater than about 20-fold increase compared to baseline. In some embodiments, the total daily dose is reduced when the level of bilirubin is greater than about 10-fold increase compared to the upper limit of normal (ULN), i.e., a “Grade 4 abnormality”. In some embodiments, where the patient has an abnormal baseline, the Grade 4 abnormality is an abnormal level of bilirubin greater than about 10-fold increase compared to baseline.

In some embodiments, the methods described herein further comprise increasing the total dose of sotorasib (e.g., from 240 mg to 480 mg, or from 480 mg to 960 mg) when liver biomarker(s) in the patient has improved to a Grade 1 or better (e.g., baseline).

### Nausea/Vomiting

In some embodiments, the adverse event is nausea or vomiting. In some embodiments, the nausea/vomiting is present despite appropriate supportive care (e.g., anti-emetic therapy). “Nausea” as used herein refers to a disorder characterized by a queasy sensation and/or the urge to vomit.

Adverse effect Grades for nausea and vomiting are defined herein by the modified Common Toxicity Criteria (CTC) provided in Table 4. See the National Cancer Institute Common Terminology Criteria for Adverse Events v5.0 (NCI CTCAE) published Nov. 27, 2017 by the National Cancer Institute, incorporated herein by reference in its entirety.

TABLE 4

| | Grade 1 | Grade 2 | Grade 3 | Grade 4 |
|---|---|---|---|---|
| Nausea | Loss of appetite | Oral intake | Inadequate oral | — |
| | without alteration | decreased without | caloric or fluid | |
| | in eating habits | significant weight | intake; tube | |
| | | loss, dehydration | feeding, total | |
| | | or malnutrition | parenteral nutrition | |
| | | | (TPN), or | |
| | | | hospitalization | |
| | | | indicated | |
| Vomiting | Intervention not | Outpatient IV | Tube feeding, TPN, | Life-threatening |
| | indicated | hydration; medical | or hospitalization | consequences |
| | | intervention | indicated | |
| | | indicated | | |

In some embodiments, the methods described herein comprise withholding sotorasib administration in a patient having ≥Grade 3 nausea until the patient has improved to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein comprise withholding sotorasib administration in a patient having ≥Grade 3 vomiting until the vomiting improves to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when nausea in the patient has improved to a Grade 1 or better (e.g., baseline).

### Diarrhea

In some embodiments, the adverse event is diarrhea. In some embodiments, the diarrhea is present despite appropriate supportive care (e.g., anti-diarrheal therapy).

Adverse effect Grades for diarrhea are defined herein by the modified Common Toxicity Criteria (CTC) provided in Table 5. See the National Cancer Institute Common Terminology Criteria for Adverse Events v5.0 (NCI CTCAE) published Nov. 27, 2017 by the National Cancer Institute, incorporated herein by reference in its entirety.

TABLE 5

| | Grade 1 | Grade 2 | Grade 3 | Grade 4 |
|---|---|---|---|---|
| Diarrhea | Increase of <4 | Increase of 4-6 | Increase of ≥7 | Life-threatening |
| | stools per day over | stools per day over | stools per day | consequences; |
| | baseline; mild | baseline; moderate | over baseline; | urgent intervention |
| | increase in ostomy | increase in ostomy | hospitalization | indicated |
| | output compared | output compared to | indicated; severe | |
| | to baseline | baseline; limiting | increase in ostomy | |
| | | instrumental | output compared to | |
| | | activities of daily | baseline; limiting | |
| | | living (ADL) | self care ADL | |

In some embodiments, the methods described herein comprise withholding sotorasib administration in a patient having ≥Grade 3 diarrhea until the patient has improved to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when diarrhea in the patient has improved to a Grade 1 or better (e.g., baseline).

### Interstitial Lung Disease

In some embodiments, the adverse event is interstitial lung disease (ILD) or pneumonitis. In cases where ILD or pneumonitis is suspected at any grade level, sotorasib is withheld. In cases where ILD or pneumonitis is confirmed, and no other causes of the ILD or pneumonitis is identified, sotorasib is permanently discontinued.

### EMBODIMENTS

1. A method of treating cancer comprising a KRAS G12C mutation in a patient comprising
(a) administering to the patient a therapeutically effective amount of sotorasib for 14 to 48 days (“an induction period”), and
(b) administering to the patient a therapeutically effective amount of sotorasib and a therapeutically effective amount of an anti-PD1 antibody or an anti-PD-L1 antibody after the induction period for the duration of a combination period.

2. The method of claim 1, wherein the therapeutically effective amount of sotorasib administered for the duration of the induction period is 960 mg.

3. The method of embodiment 1, wherein the therapeutically effective amount of sotorasib administered during the induction period is 360 mg.

4. The method of embodiment 1, wherein the therapeutically effective amount of sotorasib administered during the induction period is 240 mg.

5. The method of embodiment 1, wherein the therapeutically effective amount of sotorasib administered during the induction period is 120 mg.

6. The method of any one of embodiments 1 to 5, comprising administering the therapeutically effective amount of sotorasib to the patient once daily during the induction period.

7. The method of any one of embodiments 1 to 5, comprising administering the therapeutically effective amount of sotorasib to the patient twice daily during the induction period, wherein each dose of sotorasib corresponds to half of the therapeutically effective amount administered during the induction period.

8. The method of any one of embodiments 1 to 7, wherein the therapeutically effective amount of sotorasib administered during the combination period is 960 mg.

9. The method of any one of embodiments 1 to 7, wherein the therapeutically effective amount of sotorasib administered during the combination period is 360 mg.

10. The method of any one of embodiments 1 to 7, wherein the therapeutically effective amount of sotorasib administered during the combination period is 240 mg.

11. The method of any one of embodiments 1 to 7, wherein the therapeutically effective amount of sotorasib administered during the combination period is 120 mg.

12. The method of any one of embodiments 1 to 11, comprising administering the therapeutically effective amount of sotorasib to the patient once daily during the combination period.

13. The method of any one of embodiments 1 to 11, comprising administering the therapeutically effective amount of sotorasib to the patient twice daily during the combination period, wherein each dose of sotorasib corresponds to half of the therapeutically effective amount administered during the combination period.

14. The method of any one of embodiments 1 to 13, wherein the anti-PD-L1 antibody is atezolizumab, avelumab, or durvalumab.

15. The method of embodiment 14, wherein the anti-PD-L1 antibody is atezolizumab.

16. The method of any one of embodiments 1 to 13, wherein the anti-PD1 antibody is cemiplimab, dostarlimab, pembrolizumab, or nivolumab.

17. The method of embodiment 16, wherein the anti-PD1 antibody is pembrolizumab.

18. The method of embodiment 17, comprising administering to the patient 200 mg pembrolizumab via IV once every three weeks during the combination period.

19. The method of embodiment 1, comprising administering to the patient
360 mg sotorasib orally once per day during the induction period and the combination period; and
200 mg pembrolizumab via IV once every three weeks during the combination period.

20. The method of embodiment 1, comprising administering to the patient
960 mg sotorasib orally once per day during the induction period and the combination period; and
200 mg pembrolizumab via IV once every three weeks during the combination period.

21. The method of embodiment 1, comprising administering to the patient
240 mg sotorasib orally once per day during the induction period and the combination period; and
200 mg pembrolizumab via IV once every three weeks during the combination period.

22. The method of embodiment 1, comprising administering to the patient
120 mg sotorasib orally once per day during the induction period and the combination period and
200 mg pembrolizumab via IV once every three weeks during the combination period.

23. The method of any one of embodiments 1 to 22, wherein the induction period is 21 days.

24. The method of any one of embodiments 1 to 22, wherein the induction period is 42 days.

25. The method of any one of embodiments 1 to 24, wherein the combination period is at least 30 days.

26. The method of embodiment 25, wherein the combination period is at least 3 months.

27. The method of embodiment 26, wherein the combination period is at least 6 months.

28. The method of embodiment 27, wherein the combination period is at least 8 months.

29. The method of any one of embodiments 1 to 28, wherein the cancer exhibits a PD-L1 tumor proportion score (TPS) of 1% or greater.

30. The method of any one of embodiments 1 to 28, wherein the cancer exhibits a PD-L1 tumor proportion score (TPS) of 50% or greater.

31. The method of any one of embodiments 1 to 28, wherein the cancer exhibits a PD-L1 tumor proportion score (TPS) of 1% to 49%.

32. The method of any one of embodiments 1 to 28, wherein the cancer exhibits a PD-L1 tumor proportion score (TPS) of less than 1%.

33. The method of any one of embodiments 1 to 32, wherein the cancer is a solid tumor.

34. The method of any one of embodiments 1 to 32, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

35. The method of any one of embodiments 1 to 32, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

36. The method of any one of embodiments 1 to 32, wherein the cancer is non-small cell lung cancer.

37. The method of embodiment 36, wherein the cancer is locally-advanced or metastatic non-small cell lung cancer.

38. The method of any one of embodiments 1 to 37, wherein the patient exhibits at least a stable disease (SD), as measured by RECIST 1.1 protocol, after the combination period lasts 3, 6, or 8 months.

39. The method of any one of embodiments 1 to 37, wherein the patient exhibits at least a partial response (PR), as measured by RECIST 1.1 protocol, after the combination period lasts 3, 6, or 8 months.

40. The method of any one of embodiments 1 to 37, wherein the patient exhibits a progression free survival (PFS) of at least 3 months.

41. The method of any one of embodiments 1 to 40, wherein the patient exhibits fewer grade 3 or 4 treatment related adverse events (TRAEs) compared to a patient administered sotorasib and the anti-PD1 antibody or anti-PD-L1 antibody without an induction period.

42. The method of any one of embodiments 1 to 41, wherein the patient has not received any prior line of therapy.

43. The method of any one of embodiments 1 to 41, wherein the patient has received at least one prior line of therapy.

44. The method of any one of embodiments 1 to 43, wherein the patient has not previously received treatment with an anti-PD1 or anti-PD-L1 immunotherapy.

45. The method of any one of embodiments 1 to 43, wherein the patient has previously received treatment with anti-PD1 or anti-PD-L1 immunotherapy.

46. The method of any one of embodiments 1 to 41 and 43, wherein the patient has previously received treatment with (i) anti-PD1 or anti-PDL1 immunotherapy or (ii) prior platinum-based combination chemotherapy.

47. The method of any one of embodiments 1 to 41 and 43, wherein the patient has previous received treatment with (i) anti-PD1 or anti-PD-L1 immunotherapy and (ii) prior platinum-based combination chemotherapy.

48. The method of any one of embodiments 1 to 41 and 43 to 45, wherein the patient has previously undergone an EGFR, ALK or ROS1 targeted therapy if the cancer also exhibited a mutation in EGFR, ALK, or ROS1.

49. The method of embodiment 48, wherein the patient has progressed on an EGFR, ALK or ROS1 targeted therapy if the cancer also exhibited a mutation in EGFR, ALK, or ROS1.

50. The method of any one of embodiments 43 to 49, wherein the patient completed neoadjuvant or adjuvant chemotherapy at least 12 months prior to diagnosis of advanced stage cancer.

51. The method of embodiment any one of embodiments 1 to 41, wherein
(1) (a) the patient has previously received treatment with (i) anti-PD1 or anti-PD-L1 immunotherapy or (ii) prior platinum-based combination chemotherapy; or (b) the patient has previously received treatment with (i) anti-PD1 or anti-PD-L1 therapy and (ii) prior platinum-based chemotherapy; and
(2) the patient optionally has previously undergone an EGFR, ALK or ROS1 targeted therapy if the cancer also exhibited a mutation in EGFR, ALK, or ROS1.

52. The method of any one of embodiments 1 to 41, wherein the patient
(1) has a cancer that exhibits a PD-L1 tumor proportion score (TPS) of 50% or greater; and
(2) has not received any systemic therapy for locally advanced or metastatic non-small cell lung cancer;
   (i) but for a EGFR, ALK, or ROS1 targeted cancer therapy, if cancer exhibited a mutation in EGFR, ALK, or ROS1, and the patient has progressed on the targeted cancer therapy; and
   (ii) but for neoadjuvant or adjuvant chemotherapy completed at least 12 months prior to the start of the induction period and has not received immune checkpoint inhibitor therapy.

53. The method of any one of embodiments 1 to 41, wherein the patient
(1) has a cancer that exhibits a PD-L1 tumor proportion score (TPS) of 1% or greater; and
(2) has not received any systemic therapy for locally advanced or metastatic non-small cell lung cancer;
   (i) but for a EGFR, ALK, or ROS1 targeted cancer therapy, if cancer exhibited a mutation in EGFR, ALK, or ROS1, and the patient has progressed on the targeted cancer therapy; and
   (ii) but for neoadjuvant or adjuvant chemotherapy completed at least 12 months prior to the start of the induction period and has not received immune checkpoint inhibitor therapy.

54. The method of any one of embodiments 1 to 53, wherein the patient has an Eastern Cooperative Oncology Group (ECOG) performance status of less than or equal to 2.

55. The method of any one of embodiments 1 to 54, wherein the patient does not have active (symptomatic) brain metastases.

56. The method of any one of embodiments 1 to 55, wherein
(i) the patient
had brain metastases resected, or
received whole brain radiation therapy ending at least 4 weeks prior to start of the induction period, or
received stereotactic radiosurgery ending at least 2 weeks prior to start of the induction period, and
(ii) the patient
exhibits residual neurological symptoms of grade 2 or less, and
has not been administered steroids for at least 14 days prior to the start of the induction period, and
has an magnetic resonance imaging (MRI) performed within 14 days prior to start of the induction period that shows no evidence of progression of the brain metastases.

57. The method of any one of embodiments 1 to 56, wherein the patient does not have leptomeningeal disease.

58. The method of any one of embodiments 1 to 57, wherein the patient is not suffering from a hepatitis B infection or a hepatitis C infection.

59. The method of any one of embodiments 1 to 58, wherein the patient has not received a prior therapy with a KRAS^{G12C} inhibitor.

60. The method of embodiment 59, wherein the KRAS^{G12C} inhibitor is sotorasib or adagrasib.

61. The method of any one of embodiments 1 to 60, wherein the patient is in further need of treatment with an acid-reducing agent.

62. The method of embodiment 61, wherein the acid-reducing agent is a proton pump inhibitor (PPI), a H2 receptor antagonist (H2RA), or a locally acting antacid.

63. The method of embodiment 61 or embodiment 62, wherein the acid-reducing agent is a locally acting antacid, and wherein sotorasib is administered about 4 hours before or about 10 hours after the locally acting antacid.

64. The method of embodiment 62 or 63, wherein the locally acting antacid is sodium bicarbonate, calcium carbonate, aluminum hydroxide, or magnesium hydroxide.

65. The method of any one of embodiments 62 to 64, wherein the patient is in further need of treatment with a proton pump inhibitor (PPI) or H2 receptor antagonist (H2RA).

66. The method of embodiment 65, wherein the patient is not administered a PPI or a H2RA in combination with sotorasib.

67. The method of any one of embodiments 62, 65 or 66, wherein the PPI is omeprazole, pantoprazole, esomeprazole, lansoprazole, rabeprazole, or dexlansoprazole.

68. The method of any one of embodiments 62, 65 or 66, wherein the H2RA is famotidine, ranitidine, cimetidine, nizatidine, roxatidine or lafutidine.

69. The method of any one of embodiments 1 to 68, wherein the patient is in further need of treatment with a CYP3A4 inducer.

70. The method of embodiment 69, wherein the patient is not administered a CYP3A4 inducer in combination with sotorasib.

71. The method of embodiment 69 or 70, wherein the CYP3A4 inducer is a apalutamide, avasimibe, barbiturate, brigatinib, carbamazepine, clobazam, dabrafenib, efavirenz, elagolix, enzalutamide, eslicarbazepine, glucocorticoids, ivosidenib, letermovir, lorlatinib, lumacaftor, mitotane, modafinil, nevirapine, oritavancin, oxcarbazepine, perampanel, phenobarbital, phenytoin, pioglitazone, rifabutin, rifampin, rifapentine, St. John's wort, telotristat, or troglitazone.

72. The method of embodiment 69 or 70, wherein the CYP3A4 inducer is a strong CYP3A4 inducer.

73. The method of embodiment 72, wherein the strong CYP3A4 inducer is rifampin, mitotane, avasimibe, rifapentine, apalutamide, ivosidenib, phenytoin, carbamazepine, enzalutamide, St John's Wort extract, or lumacaftor.

74. The method of any one of embodiments 1 to 73, wherein the patient is in further need of treatment with a CYP3A4 substrate.

75. The method of embodiment 74, wherein the patient is not administered a CYP3A4 substrate in combination with sotorasib.

76. The method of embodiment 72 or 73, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus (fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, or ziprasidone.

77. The method of embodiment 74 or 75, wherein the CYP3A4 substrate is a CYP3A4 substrate with a narrow therapeutic index.

78. The method of embodiment 77, wherein the CYP3A4 substrate with a narrow therapeutic index is alfentanil, cyclosporine, dihydroergotamine, ergotamine, everolimus, fentanyl, primozide, quinidine, tacrolimus, or sirolimus.

79. The method of any one of embodiments 1 to 78, wherein the patient is in further need of treatment with a P-glycoprotein (P-gp) substrate.

80. The method of embodiment 79, wherein the patient is not administered a P-gp substrate in combination sotorasib.

81. The method of embodiment 79 or 80, wherein the P-gp substrate is etexilate, digoxin, fexofenadine, everolimus, cyclosporine, sirolimus, tacrolimus, or vincristine.

82. The method of embodiment 79 or 80, wherein the P-gp substrate is a P-gp substrate with a narrow therapeutic index.

83. The method of embodiment 82, where in the P-gp substrate with a narrow therapeutic index is digoxin, everolimus, cyclosporine, tacrolimus, sirolimus, or vincristine.

### EXAMPLES

### Example 1—Sotorasib and Pembrolizumab

This example describes a phase 1b, multicenter, open label study evaluating the safety, tolerability, pharmacokinetics, and efficacy of sotorasib in combination with pembrolizumab in subjects with KRAS p.G12C mutant non-small cell lung cancer (NSCLC). Sotorasib will be administered orally daily, and pembrolizumab will be administered intravenously. The dose of pembrolizumab will be its approved dose (200 mg Q3W) in NSCLC while sotorasib dose will vary across different Dose Levels. The terms “subject” and “patient” are used interchangeably herein.

### Part 1:

Part 1 of the study will assess the safety of sotorasib in combination with pembrolizumab. Part 1 will enroll up to 40 subjects and will consist of 2 cohorts (Cohort A and Cohort B) based on the timing of initiation of pembrolizumab as described below. Both Cohort A and B will begin enrollment at the same time.

### Part 1 Cohort A:

In Cohort A, sotorasib will be administered daily from day 1 and pembrolizumab Q3W will be administered starting from day 22.

The planned dose levels for sotorasib in combination with a fixed dose of pembrolizumab (200 mg) are:
Dose Cohort Level A1: Sotorasib 360 mg QD
Dose Cohort Level A2: Sotorasib 960 mg QD.
Dose Cohort Level A-1: Sotorasib 240 mg QD
Dose Cohort Level A-2: Sotorasib 120 mg QD

Dose exploration will begin with 3 to 6 subjects treated at Dose Cohort Level A1. The study DLT period starts on day 22 and continues to day 42. Once at least 3 subjects enrolled at a certain Dose Cohort Level are DLT evaluable, a dose level review team (DLRT) meeting will be convened. Depending on observed safety data, the following may occur: 1) additional enrollment to Dose Cohort Level A1, up to a maximum of 10 evaluable subjects, or 2) dose escalation to Dose Cohort Level A2, or 3) dose de-escalation to Dose Cohort Level A-1.

### Part 1 Cohort B:

In Cohort B, sotorasib will be administered daily starting from day 1 and pembrolizumab will be administered Q3W starting from day 43.

The planned dose levels for sotorasib in combination with a fixed dose of pembrolizumab (200 mg) are:
Dose Cohort Level B1: Sotorasib 360 mg QD.
Dose Cohort Level B2: Sotorasib 960 mg QD
Dose Cohort Level B-1: Sotorasib 240 mg QD
Dose Cohort Level B-2: Sotorasib 120 mg QD

Dose exploration will begin with 3 to 6 subjects treated at Dose Cohort Level B1. The study DLT period starts on day 43 and continues to day 63. Once at least 3 subjects enrolled at a certain Dose Cohort Level are DLT evaluable, a DLRT meeting will be convened.

Depending on observed safety data, the following may occur: 1) additional enrollment to Dose Cohort Level B1, up to a maximum of 10 evaluable subjects, or 2) dose escalation to Dose Cohort Level B2, or 3) dose de-escalation to Dose Cohort Level B-1.

Rules for dose de-escalation/re-escalation are derived using a modified Toxicity Probability Interval-2 (mTPI-2) model (Guo et al, 2017) with a target toxicity probability of 0.30, as shown in the below Table 6.

TABLE 6

| No. of | Number of Subjects | |
|---|---|---|
| Evaluable^{a} | with DLT observed at | |
| Subjects | current dose level | Guideline for Dose Escalation/De-Escalation |
| 3 | 0 | Escalate Dose Level^{b} |
| 3 | 1 | Stay at current dose level |
| 3 | ≥2 | De-escalate or stop enrollment at current dose level^{b} |
| 4 | 0 | Escalate Dose Level^{b} |
| 4 | 1 | Stay at current dose level |
| 4 | ≥2 | De-escalate or stop enrollment at current dose level^{b} |
| 5 | ≤1 | Escalate Dose Level^{b} |
| 5 | ≥2 | De-escalate or stop enrollment at current dose level^{b} |
| 6 | ≤1 | Escalate Dose Level^{b} |
| 6 | ≥2 | De-escalate or stop enrollment at current dose level^{b} |
| 7 or 8 | ≤1 | Escalate Dose Level^{b} |
| 7 or 8 | 2 | Stay at current dose level |
| 7 or 8 | ≥3 | De-escalate or stop enrollment at current dose level^{b} |
| 9 | ≤2 | Escalate Dose Level^{b} |
| 9 | ≥3 | De-escalate or stop enrollment at current dose level^{b} |
| 10 | ≤2 | Escalate Dose Level^{b} |
| 10 | 3 | Enrollment to current Dose Level is complete |
| 10 | ≥4 | De-escalate^{b} |

DLT = dose-limiting toxicity
^{a}A subject is evaluable if subject experiences a DLT or completes 42 days on treatment (Cohort A) or 63 days on treatment (Cohort B) and receives ≥80 of planned cumulative dose of sotorasib and one full planned dose of pembrolizumab during the DLT evaluation window. A subject will be DLT evaluable if he/she is available for follow up during the 21-day DLT period.
^{b}De-escalate guideline applies only when enrollment is allowed to a lower dose level. Re-escalation to the next higher Dose Level may be allowed, as appropriate, only in the following instances: 1) 2 of 5 or 2 of 6 evaluable subjects experience a DLT or 2) 3 of 8 or 3 of 9 subjects experience a DLT.

In addition to the planned dose levels, further degree of dose modification (e.g., intermediate doses) and/or schedule of administration (e.g., twice daily [BID] dosing) will be determined based on analysis of emerging safety and PK data. The maximum tolerated dose (MTD) will be estimated using isotonic regression (Ji et al, 2010) and the MTD will be the dose level with the estimated DLT rate closest to 0.30. No more than 10 DLT evaluable subjects will be enrolled at any specific dose level in Part 1.

### Part 1 Will End Once any of the Following Events Occur:

Any dose level (or intermediate dose level) in Cohorts A and B is deemed safe and tolerable (minimum 6 evaluable subjects at that Dose Level) and the next higher dose level (if any) in that Cohort is deemed unsafe and intolerable.
All dose levels (including any intermediate dose levels) in Cohort A and B are deemed unsafe and intolerable.
On the basis of a review of real-time safety data and available preliminary PK data, dose escalation may be halted or modified by the Sponsor as deemed appropriate.

### Part 2:

Once the dose and dosing schedule deemed safe and tolerable in any Cohort in Part 1 is identified by the DLRT and depending on the data obtained, enrollment may commence in the dose expansion phase (Part 2) in either Cohort A or Cohort B to confirm the safety and tolerability of that Dose Level and to further evaluate anti-tumor activity.

If more than one Dose Level is deemed safe and tolerable in Part 1 Cohort A and Cohort B, the sponsor in consultation with DLRT will determine the dose and schedule to be implemented in Part 2. Only one Cohort in Part 2 will be opened for enrollment. Part 2 will enroll up to 20 subjects.
Part 2 Cohort A—Sotorasib (dose and schedule deemed safe and tolerable from Part 1 Cohort A) and pembrolizumab 200 mg Q3W starting from day 22

### OR

Part 2 Cohort B—Sotorasib (dose and schedule deemed safe and tolerable from Part 1 Cohort B) and pembrolizumab 200 mg Q3W starting from day 43

Interim safety analyses will be done after n=10 dose expansion subjects have had the opportunity to be on treatment for at least 21 days since first dose of pembrolizumab. Based on these interim safety results and after reviewing the updated estimate of the MTD or recommended safe combination dose using all available data including data from dose exploration and expansion subjects, the DLRT may modify the dose level of treated subjects. Intermediate dose levels and/or alternative dosing schedules may be explored. A final estimate of the MTD or recommended safe combination will use all data from dose exploration and dose expansion.

For both Part 1 and Part 2, administration of sotorasib and pembrolizumab may continue until evidence of disease progression, intolerance to study medication, withdrawal of consent, or end of study. In case of intolerance or toxicity to pembrolizumab, the subject may continue on sotorasib alone until evidence of disease progression, withdrawal of consent or end of study. Subjects who permanently discontinue sotorasib will also be discontinued from treatment with pembrolizumab. Subjects with radiological progression who continue to derive unequivocal clinical benefit in the judgement of the Investigator may continue on treatment after discussion with the Medical monitor. These subjects must have a radiological evaluation every 6 to 8 weeks while on treatment.

### Endpoints:

| | |
|---|---|
| Objectives | Endpoints |

Primary

| | |
|---|---|
| To evaluate the safety and tolerability of | Dose-limiting toxicities (DLTs), |
| sotorasib in combination with | treatment-emergent adverse events, |
| pembrolizumab in adult subjects with | treatment-related adverse events, and |
| KRAS p.G12C mutant advanced non- | changes in vital signs, electrocardiograms |
| small cell lung cancer (NSCLC) | (ECGs), and clinical laboratory tests |

Secondary

| | |
|---|---|
| To characterize pharmacokinetics (PK) | Pharmacokinetic parameters of product(s) |
| of sotorasib in combination with | including, but not limited to, maximum |
| pembrolizumab in adult subjects with | plasma concentration (Cₘₐₓ), time to |
| KRAS p.G12C mutant advanced | maximum plasma concentration (tₘₐₓ), |
| NSCLC. | and area under the plasma |
| | concentration-time curve (AUC) |
| To evaluate anti-tumor activity of | Objective response rate (ORR), disease |
| sotorasib in combination with | control rate, duration of response, time to |
| pembrolizumab in adult subjects with | response, progression-free survival |
| KRAS p.G12C mutant NSCLC | measured by computed tomography (CT) |
| | or magnetic resonance imaging (MRI) and |
| | assessed per Response Evaluation |
| | Criteria in Solid Tumors Version 1.1 |
| | (RECIST 1.1), and overall survival (OS). |

Exploratory

| | |
|---|---|
| To explore PK/pharmacodynamic | Sotorasib and pembrolizumab |
| (PD) relationships for safety | exposure/safety and |
| and/or efficacy endpoints | exposure/efficacy |
| | relationships |
| To investigate potential biomarkers by | Quantification of biomarker |
| biochemical and/or genetic analysis of | expression at protein, RNA, |
| blood and/or tumor tissue samples. | and DNA levels, as |
| | appropriate in blood |
| | and/or tumor samples |

### Inclusion Criteria:

Subjects are eligible to be included in the study only if all of the following criteria apply:

Subject has provided informed consent prior to initiation of any study specific activities/procedures.

Age ≥18 years.

Pathologically documented, uncurable locally-advanced or metastatic NSCLC KRAS p.G12C mutation identified through molecular testing. KRAS p.G12C mutation must be identified by an approved diagnostic device for detection of KRAS p.G12C in NSCLC or be performed in a Clinical Laboratory Improvement Amendments (CLIA) certified laboratory in the United States.

For Part 1, subjects must have received anti-PD1 or anti PD-L1 immunotherapy (unless contraindicated) and/or platinum-based combination chemotherapy and targeted therapy (if actionable oncogenic driver mutations were identified [eg, EGFR, ALK, and ROS1]), unless they refused standard therapy. Prior neoadjuvant/adjuvant chemotherapy will be considered for eligibility only if the subject progressed on or within 6 months of completion of the therapy.

For Part 2, subjects must have PD-L1 expression (TPS>50%, or TPS≥1%) and must not receive systemic therapy for uncurable locally advanced or metastatic NSCLC; subjects with known actionable mutations, as per local standards (such as EGFR, ALK, but not limited to) must have progressed on standard of care targeted therapy.

Note: Prior neoadjuvant or adjuvant chemotherapy with curative intent will be allowed if it was completed at least 12 months prior to the diagnosis of advanced stage disease and subject has no prior history of immune checkpoint inhibitor therapy.

Subjects must be willing to undergo pretreatment tumor biopsy if clinically feasible. If a tumor biopsy prior to treatment is not clinically feasible, or if the sample has insufficient tissue for testing, subjects must be willing to provide archived tumor tissue samples (formalin-fixed, paraffin-embedded [FFPE] sample collected within 5 years). Subjects who do not have archived tissue available can be allowed to enroll without undergoing tumor biopsy upon agreement with investigator and the Medical Monitor if a tumor biopsy is not feasible.

Measurable disease per (response evaluation criteria in solid tumors (RECIST) 1.1 criteria. Lesions previously radiated are not considered measurable unless they have progressed after radiation.

Eastern Cooperative Oncology Group (ECOG) Performance Status of ≤2.

Life expectancy of >3 months, in the opinion of the investigator.

Ability to take oral medications and willing to record daily adherence to investigational product.

Corrected QT interval (QTc)≤470 msec for females and QTc≤450 msec for males (based on average of screening triplicates).

Adequate hematological laboratory assessments, defined as the following within 10 days prior to start of study therapy
Absolute neutrophil count (ANC) ≥1500 cells/μL
Hemoglobin ≥9.0 g/dL
Platelet count ≥100000/μL

Adequate renal laboratory assessments, defined as the following: Estimated glomerular filtration rate based on Modification of Diet in Renal Disease (MDRD) calculation ≥60 ml/min/1.73 m².

Adequate hepatic laboratory assessments, as follows:
Aspartate aminotransferase (AST)≤2.5× upper limit of normal (ULN)
Total bilirubin (TBL)≤1.5×ULN
   For subjects with known Gilbert's syndrome, total bilirubin up to 2.0×ULN will be allowed if their AST, ALT, and direct bilirubin are within normal limits.

Adequate coagulation laboratory assessments, as follows:
Prothrombin time (PT) or partial thromboplastin time (PTT)<1.5×ULN, OR
International normalized ratio (INR)<1.5×ULN or within target range if on prophylactic anticoagulation therapy

### Exclusion Criteria

Subjects are excluded from the study if any of the following criteria apply:

Mixed small-cell lung cancer and NSCLC histology

Symptomatic spinal cord compression or active (symptomatic) brain metastases, EXCEPT

Subjects with asymptomatic brain metastases are eligible if the treating physician determines that immediate central nervous system (CNS)-specific treatment is not required and is unlikely to be required during the first cycle of study drug.

Subjects with treated brain metastases, who have had brain metastases resected or have received whole brain radiation therapy ending at least 4 weeks (or stereotactic radiosurgery ending at least 2 weeks) prior to study day 1 are eligible if they meet all of the following criteria: a) residual neurological symptoms grade ≤2; b) off steroids for at least 14 days prior to cycle 1 day 1; and c) follow-up Magnetic Resonance Imaging (MRI) performed within at least 14 days prior to cycle 1 day 1 shows no evidence of progression. For determining the grade of any neurological symptom attributable to brain metastases, see National Cancer Institute Common Terminology Criteria for Adverse Events v5.0 (NCI CTCAE) published Nov. 27, 2017 by the National Cancer Institute, incorporated herein by reference in its entirety.

### Leptomeningeal Disease

Uncontrolled pleural effusion, pericardial effusion, or ascites requiring recurrent drainage procedures at a frequency greater than monthly. Patients with PleurX catheters in place may be considered for the study only with Medical Monitor approval.

History or presence of hematological malignancies unless curatively treated with no evidence of disease ≥3 years.

History of other malignancy within the past 2 years, with the following exceptions:
Malignancy treated with curative intent and with no known active disease present for ≥2 years before enrollment and felt to be at low risk for recurrence by the treating physician.
Adequately treated non-melanoma skin cancer or lentigo maligna without evidence of disease.
Adequately treated cervical carcinoma in situ without evidence of disease.
Adequately treated breast ductal carcinoma in situ without evidence of disease.
Prostatic intraepithelial neoplasia without evidence of prostate cancer.
Adequately treated urothelial papillary noninvasive carcinoma or carcinoma in situ.

Myocardial infarction within 6 months of study day 1, symptomatic congestive heart failure (New York Heart Association [NYHA]>class II), unstable angina, or any clinically significant abnormalities on resting electrocardiogram (ECG).

Major surgery within 28 days of study day 1.

Gastrointestinal (GI) tract disease causing the inability to take oral medication, malabsorption syndrome, requirement for intravenous (IV) alimentation, uncontrolled inflammatory GI disease (e.g., Crohn's disease, ulcerative colitis).

Evidence of hepatitis infection based on the following results and/or criteria:
Positive Hepatitis B Surface Antigen (HepBsAg) (indicative of chronic Hepatitis B or recent acute hepatitis B).
Negative HepBsAg with a positive for hepatitis B core antibody (Hepatitis B core antibody testing is not required for screening, however if this is done and is positive, then hepatitis B surface antibody [Anti-HBs] testing is necessary. Undetectable anti-HBs in this setting would suggest unclear and possible infection, and needs exclusion).
Positive Hepatitis C virus antibody: Positive Hepatitis C antibody with evidence of Hepatitis C RNA by polymerase chain reaction (PCR).

Positive test for human immunodeficiency virus (HIV).

Has a diagnosis of immunodeficiency or is receiving chronic systemic steroid therapy (in dosing exceeding 10 mg daily of prednisone or equivalent) or any other form of immunosuppressive therapy within 7 days prior the first dose of study drug.

Is on chronic systemic steroids. Subjects with asthma that require intermittent use of bronchodilators, inhaled steroids, or local steroid injections would not be excluded from the study.

Has an active autoimmune disease that has required systemic treatment in past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) is not considered a form of systemic treatment and is allowed.

Has a history of (non-infectious) pneumonitis that required steroids or has current pneumonitis.

Has an active infection requiring systemic therapy.

Therapeutic oral or intravenous antibiotics within 2 weeks prior to study day 1. Prophylactic antibiotics are allowed with sponsor medical monitor approval.

Current Common Terminology Criteria for Adverse Events (CTCAE) version 5.0 grade ≥2 peripheral neuropathy.

Anti-tumor therapy (chemotherapy, antibody therapy, molecular targeted therapy, or investigational agent) within 28 days of study day 1. Exception: Subjects who receive prior small molecule targeted therapy within 14 days of study day 1.

Therapeutic or palliative radiation therapy within 2 weeks of study day 1. Subjects must have recovered from all radiotherapy related toxicity to grade 1 or better.

Received radiation therapy to the lung that is >30 Gy within 6 months of the first dose of trial treatment.

Participating in any other interventional trial(s).

Previous treatment with a KRAS p.G12C inhibitor.

Use of known cytochrome P450 (CYP) 3A4 sensitive substrates with a narrow therapeutic window (e.g., alfentanil, cyclosporine, dihydroergotamine, ergotamine, everolimus, fentanyl, primozide, quinidine, tacrolimus, or sirolimus) or P-gp substrates (digoxin, everolimus, cyclosporine, tacrolimus, sirolimus, or vincristine) within 14 days or 5 half-lives of the drug or its major active metabolite, whichever is longer, prior to study day 1 that was not reviewed and approved by the principal investigator and the sponsor medical monitor.

Use of strong inducers of CYP3A4 (including herbal supplements such as St. John's wort) (e.g., rifampin, mitotane, avasimibe, rifapentine, apalutamide, ivosidenib, phenytoin, carbamazepine, enzalutamide, St John's Wort extract, or lumacaftor) within 14 days or 5 half-lives (whichever is longer) prior to study day 1 that was not reviewed and approved by the principal investigator and the sponsor medical monitor.

Unresolved toxicities from prior anti-tumor therapy, defined as not having resolved to CTCAE version 5.0 grade 0 or 1, or to levels dictated in the eligibility criteria with the exception of alopecia (any grade allowed). Grade 2 or 3 toxicities from prior anti-tumor therapy that are considered irreversible (defined as having been present and stable for >6 months), such as ifosfamide related proteinuria or neuropathy, may be allowed if they are not otherwise described in the exclusion criteria AND there is agreement to allow by both the investigator and sponsor.

Subject unable to receive both iodinated contrast for computed tomography (CT) scans and gadolinium contrast for MRI scans.

Has received a live vaccine within 30 days prior to the first dose of study drug. Examples of live vaccines include, but are not limited to, the following: measles, mumps, rubella, varicella/zoster (chicken pox), yellow fever, rabies, Bacillus Calmette-Guérin (BCG), and typhoid vaccine. Seasonal influenza vaccines for injection are generally killed virus vaccines and are allowed; however, intranasal influenza vaccines (e.g., FluMist®) are live attenuated vaccines and are not allowed.

Currently receiving treatment in another investigational device or drug study, or less than 28 days since ending treatment on another investigational device or drug study(ies). Other investigational procedures while participating in this study are excluded.

Has received prior therapy with an anti-PD-1, anti-PD-L1, or anti-PD-L2 agent and was discontinued from that treatment due to an immune-mediated adverse event.

Subject has known sensitivity to any of the products or components to be administered during dosing.

Subject likely to not be available to complete all protocol-required study visits or procedures, and/or to comply with all required study procedures to the best of the subject and investigator's awareness.

History or evidence of any other clinically significant disorder, condition or disease (with the exception of those outlined above) that, in the opinion of the investigator or sponsor physician, if consulted, would pose a risk to subject safety or interfere with the study evaluation, procedures or completion.

Female subjects of childbearing potential with a positive pregnancy test assessed at Screening or day 1 by a serum pregnancy test and/or urine pregnancy test.

Female subject is pregnant or lactating/breastfeeding or planning to become pregnant or breastfeed during treatment and for an additional: 7 days after the last dose of sotorasib OR 4 months after the last dose of pembrolizumab.

Female subjects of childbearing potential unwilling to use 1 highly effective method of contraception during treatment and for an additional: 7 days after the last dose of sotorasib OR 4 months after the last dose of pembrolizumab.

Female subjects of childbearing potential unwilling to use 1 highly effective method of contraception during treatment and for an additional 4 months after receiving the last dose of pembrolizumab.

Male subjects with a female partner of childbearing potential who are unwilling to practice sexual abstinence (refrain from heterosexual intercourse) or use contraception during treatment and for an additional 7 days after the last dose of sotorasib.

Male subjects with a pregnant partner who are unwilling to practice abstinence or use a condom during treatment and for an additional 7 days after the last dose of sotorasib.

Male subjects unwilling to abstain from donating sperm during treatment and for an additional 7 days after the last dose of sotorasib.

### Dosing Instructions

Sotorasib: Sotorasib will be administered orally daily (QD) with or without food for a treatment cycle of 21 days.

Subject should take the sotorasib dose (all tablets at the same time) at approximately the same time every day. The sotorasib dose should also not be taken more than 2 hours earlier than the target time based on previous day's dose. The sotorasib dose should not be taken more than 6 hours after the dosing time. Take the next dose as prescribed. If vomiting occurs after taking sotorasib, do not take an additional dose. Take the next dose as prescribed.

Administration to Patients Who Have Difficulty Swallowing Solids: Disperse tablets in 120 mL (4 ounces) of non-carbonated, room-temperature water without crushing. No other liquids should be used. Stir until tablets are dispersed into small pieces (the tablets will not completely dissolve) and drink immediately or within 2 hours. The appearance of the mixture may range from pale yellow to bright yellow. Swallow the tablet dispersion. Do not chew pieces of the tablet. Rinse the container with an additional 120 mL (4 ounces) of water and drink. If the mixture is not consumed immediately, stir the mixture again to ensure that tablets are dispersed.

Pembrolizumab: The recommended dose of pembrolizumab is 200 mg administered as an IV infusion over 30 minutes Q3W. Administer diluted solution intravenously over 30 minutes through an intravenous line containing a sterile, non-pyrogenic, low-protein binding, 0.2 to 5 micron in-line or add-on filter.

### Dose Limiting Toxicities (DLTs)

The DLT window (i.e., DLT-evaluable period) will start on day 22 and continues to day 42 of sotorasib and pembrolizumab treatment for subjects enrolled in Part 1 Cohort A, and start on day 43 and continues to day 63 for subjects enrolled in Part 1 Cohort B. The grading of adverse events will be based on the guidelines provided in the CTCAE version 5.0. A subject will be DLT evaluable if the subject has completed the DLT window as described above and received ≥80% of the planned cumulative dose of sotorasib and one full dose of pembrolizumab or experienced a DLT any time during the DLT window. A subject will be DLT evaluable if he/she is available for follow-up during the 21-day DLT period.

Dose-limiting toxicity is defined as any adverse event meeting the criteria listed below occurring during the DLT evaluation window and related to sotorasib or pembrolizumab.

An adverse event that results in permanent discontinuation of any investigational product (except discontinuation for infusional reaction for pembrolizumab).
Febrile neutropenia.
Grade 4 neutropenia of any duration.
Grade 3 neutropenia lasting >7 days.
Grade 3 thrombocytopenia for >7 days.
Grade 3 thrombocytopenia with grade ≥2 bleeding.
Grade 4 thrombocytopenia.
Grade 4 anemia.
Grade ≥4, vomiting or diarrhea.
Grade 3 vomiting or Grade 3 diarrhea lasting more than 3 days despite optimal medical support.
Grade ≥3 nausea lasting 3 days or more despite optimal medical support.
Grade ≥3 ALT or AST elevations.
Grade ≥3 bilirubin elevation.

Any other grade ≥3 adverse event including grade ≥3 immune-mediated adverse event with the following exceptions: fatigue; Grade 3 or Grade 4 immune-mediated endocrinopathy that can be managed with replacement therapy; Grade 3 immune-mediated colitis; Asymptomatic Grade 3 electrolyte abnormalities that last <72 hours, are not clinically complicated, and resolve spontaneously or respond to medical interventions; Grade 3 amylase or lipase that is not associated with symptoms or clinical manifestations of pancreatitis; Other select lab abnormalities that do not appear to be clinically relevant or harmful to the patient and/or can be corrected with replacement or modifications (e.g., grade 3 lymphopenia, grade 3 hypoalbuminemia).

Any subject meeting the criteria for Hy's Law case (i.e., severe drug-induced liver injury [DILI]) will be considered a DLT. A Hy's Law case is defined as: AST or ALT values of ≥3×ULN AND with serum TBL of >2×ULN without signs of cholestasis and with no other clear alternative reason to explain the observed liver-related laboratory abnormalities.

### Dose Level Determination:

A recommendation to escalate to a higher dose cohort will only occur when the previous dose regimen(s) has been found to be reasonably tolerated based on available study data and after recommendation by DLRT voting members. Available data from previous cohorts will also be considered. Dose level recommendations will be made on a treatment cohort basis (not on an individual basis). After receiving the DLRT recommendation, sponsor will render a final decision and will issue a written notification of the dose change decision to investigators.

### Dose Adjustments, Delays, Rules for Withholding or Restarting, Permanent Discontinuation:

### Sotorasib:

Dose reduction levels of sotorasib for toxicity management of individual subjects are provided in TABLE 7. Sotorasib will be discontinued, or dosage reduced, in the event of a toxicity that, in the opinion of the investigator, warrants the discontinuation, or dose reduction as indicated in TABLE 8. Dose reductions below 120 mg are not allowed. Subjects who experience an adverse event requiring dose reductions below 120 mg should be permanently discontinued from sotorasib treatment.

TABLE 7
DOSE REDUCTIONS FOR SOTORASIB
Sotorasib Dose Levels (mg)

| Dose 0 | Dose −1 | Dose −2 | Dose −3 | Dose −4 | Dose −5 |
|---|---|---|---|---|---|
| 960 | 720 | 480 | 360 | 240 | 120 |

TABLE 8
SOTORASIB DOSE MODIFICATION GUIDELINES FOR HEMATOLOGICAL
AND NON-HEMATOLOGICAL TOXICITIES

| | Recommended Action | |
|---|---|---|
| Toxicity | Hold Until: | Restart Dose: |
| Grade ≥3 nausea, vomiting, or | Recovery to grade 1 or less or to | Resume dosing at 1 dose |
| diarrhea lasting longer than | baseline grade | lower^{a} |
| 3 days despite optimal medical | | |
| support | | |
| Any other drug-related | Recovery to grade 1 or less or to | Resume dosing at 1 dose |
| toxicity ≥grade 3^{b} | baseline grade ≤4 weeks from | lower^{a} |
| | toxicity event | |
| | Recovery to grade 1 or less or to | Resume dosing at 2 doses |
| | baseline grade >4 weeks from | lower^{a} |
| | toxicity event | |
| Suspected interstitial lung disease | ILD/pneumonitis confirmed or | If confirmed, permanently |
| (ILD)/pneumonitis of any grade | excluded | discontinue sotorasib |
| | | If excluded, restart at same |
| | | dose if no other sotorasib |
| | | dose modification guidelines |
| | | are applicable |

^{a}Subjects may be resumed at a dose lower than the recommended restarting dose after discussion with the medical monitor.
^{b}For suspected hepatotoxicity, refer to section “Hepatotoxicity Stopping and Rechallenge”, below, and as discussed in Guidance for Industry Drug-Induced Liver Injury: Premarketing Clinical Evaluation, July 2009.

### Pembrolizumab:

No dose reductions of pembrolizumab are recommended. Guidelines for withholding and permanent discontinuation of pembrolizumab are listed in TABLE 9. For guidelines on management of specific immune-mediated adverse events, please refer to the KEYTRUDA® U.S. Prescribing Information, Merck & Co., Inc., Whitehouse Station, New Jersey, 08889 (revision February 2022), incorporated herein by reference in its entirety.

TABLE 9

| Adverse Reaction | Severity^{a} | Dose Modification |
|---|---|---|
| Immune-mediated | Grade 2 | Withhold^{b} |
| pneumonitis | Grades 3 or 4 or recurrent Grade 2 | Permanently discontinue |
| Immune-mediated | Grades 2 or 3 | Withhold^{b} |
| colitis | Grade 4 | Permanently Discontinue |
| Immune-mediated | AST or ALT >3 but no more than 5 × ULN or total | Withhold^{c} |
| hepatitis | bilirubin >1.5 but no more than 3 × ULN | |
| | In patients without liver metastases, AST or | Permanently discontinue |
| | ALT >5 × ULN or total bilirubin >3 × ULN | |
| | In patients with liver metastasis and Grade 2 | |
| | AST or ALT at baseline, with an increase in AST | |
| | or ALT of 50% or more relative to baseline that | |
| | persists for at least 1 week | |
| Immune-mediated | Grades 3 or 4 | Withhold until clinically |
| endocrinopathies | | stable |
| Immune-mediated | Grade 2 | Withhold until clinically |
| nephritis | | stable |
| | Grades 3 and 4 | Withhold^{b} |
| Immune-mediated skin | Grade 3 or suspected Stevens-Johnson Syndrome | Withhold |
| adverse reactions | (SJS) or toxic epidermal necrolysis (TEN) | |
| | Grade 4 or confirmed SJS or TEN | Permanently discontinue |
| Other immune-mediated | Grades 2 and 3 based on the severity and type of | Withhold^{b} |
| adverse reactions | reaction | |
| | Grade 3 based on the severity and type of reaction or | Permanently discontinue |
| | Grade 4 | |
| Recurrent | Recurrent Grade 2 pneumonitis | Permanently discontinue |
| immune-mediated | Recurrent Grades 3 or 4 | |
| adverse reactions | | |
| Inability to taper | Requirement for 10 mg per day or greater prednisone | Permanently discontinue |
| corticosteroid | or equivalent for more than 12 weeks after last dose | |
| | of KEYTRUDA | |
| Persistent Grade 2 or 3 | Grades 2 or 3 adverse reactions lasting 12 weeks or | Permanently discontinue |
| adverse reaction | longer after last dose of KEYTRUDA | |
| (excluding | | |
| endocrinopathy) | | |
| Infusion-related | Grades 1 or 2 | Interrupt or slow the rate |
| reactions | | of infusion |
| | Grades 3 or 4 | Permanently discontinue |

ALT = alanine transaminase;
AST = aspartate transaminase;
CRF = case report form;
CTCAE = Common Terminology Criteria for Adverse Events;
ULN = upper limit of normal
KEYTRUDA ® US Prescribing Information, 2020
^{a}Toxicity was graded per National Cancer Institute Common Terminology Criteria for Adverse Events Version 4.0 (NCI CTCAE v4).
^{b}Resume in patients with complete or partial resolution (Grades 0 to 1) after corticosteroid taper.
^{c}If ALT or AST ≥10 × ULN or >3 × ULN with concurrent total bilirubin ≥2 × ULN, permanently discontinue KEYTRUDA and consider corticosteroid therapy

### Hepatotoxicity Stopping and Rechallenge Rules:

Guidelines for management and monitoring of subjects with increased AST, ALT, or alkaline phosphatase ALP) related to sotorasib are outlined in TABLE 10.

TABLE 10
If the conditions for permanent discontinuation are met (below): Subject to be permanently
discontinued AST or ALT >3 × ULN and INR >1.5 × ULN (for subjects not on anticoagulation
therapy) in the presence of no important alternative causes for elevated AST/ALT values OR
AST or ALT >3 × ULN and TBL >2 × ULN in the presence of no important alternative causes
for elevated AST/ALT and/or TBL values
If conditions are not met: Exclude other causes^{a}
Upon failure to identify any other causes and sotorasib relation to increase in LFTs cannot
be excluded, proceed with guidelines below:

| | Sotorasib | Medical | |
|---|---|---|---|
| CTCAE Grade | Action | Management | Monitoring and Follow-up |
| Grade 2 AST or ALT | Withhold | Initiate steroids^{b} | Closely monitor liver function tests |
| and ALP ≤8 × ULN | | | Await resolution to baseline or grade 1 |
| with no clinical | | | Restart at same dose level |
| symptoms consistent | | | |
| with hepatitis | | | |
| (right upper quadrant | | | |
| pain/tenderness, | | | |
| fever, nausea, | | | |
| vomiting, and | | | |
| jaundice) | | | |
| Grade 2 AST or ALT | First | Initiate steroids^{b} | Closely monitor liver function tests |
| with symptoms | Occurrence | | Await resolution to baseline or grade 1 |
| Or | Withhold | | and resolution or improvement of |
| Grade 3 or 4 | | | hepatitis symptoms |
| AST or ALT | | | Restart at 2 dose level reduction^{c, e} |
| Or | Second | Initiate steroids^{b} | Closely monitor liver function tests |
| 8 × ULN ALP^{d} | Occurrence | | Await resolution to baseline or grade 1 |
| | Withhold | | and resolution or improvement of |
| | | | hepatitis symptoms |
| | | | Resume at an additional 2 dose level |
| | | | reduction only with MEDICAL |
| | | | MONITOR approval^{c, e} |

| | |
|---|---|
| Third | NOT APPLICABLE |
| Occurrence | |
| Permanently | |
| discontinue | |
| sotorasib | |

ALP = alkaline phosphatase;
ALT = alanine aminotransferase;
AST = aspartate aminotransferase;
CTCAE = Common Terminology Criteria for Adverse Events;
INR = international normalized ratio;
LFT = liver function test;
TBL = total bilirubin;
ULN = upper limit of normal
^{a}If increase in AST/ALT is likely related to alternative agent, discontinue causative agent and await resolution to baseline or grade 1 prior to resuming sotorasib.
^{b}For example: prednisone 1.0 to 2.0 mg/kg/day, dexamethasone equivalent, or methylprednisolone equivalent, followed by a taper. The taper may occur after restarting sotorasib.
^{c}Close monitoring at restart (e.g., daily LFTs × 2, then weekly × 4). Sotorasib dose may be increased after discussion with medical monitor.
^{d}There is no limit to the number of sotorasib re-challenges for isolated alkaline phosphatase elevations that resolve to baseline or grade 1.
^{e}Dose decrements below 120 mg are not allowable. If AST/ALT levels are beyond ULOQ, as additional serum samples should be collected.

### Efficacy Assessments

The extent of disease will be evaluated by contrast-enhanced MRI/CT according to RECIST 1.1. All radiological imaging will be performed as indicated in the Site Imaging Manual provided by the central imaging core laboratory. In order to reduce radiation exposure for subjects, the lowest dose possible should be utilized whenever possible.

The screening scans must be performed within 28 days prior to start of treatment and will be used as baseline. Imaging performed as part of standard of care that falls within the 28 day screening window given for scans may be used for the baseline scan as long as it meets the scan requirements for screening. All subsequent scans will be performed in the same manner as at screening, with the same contrast, preferably on the same scanner. Radiological assessment must include CT of the chest, and contrast enhanced CT or MRI of the abdomen and pelvis, as well as assessment of all other known sites of disease as detailed within the Site Imaging Manual.

The same imaging modality, MRI field strength and IV and oral contrast agents should be used at screening and for all subsequent assessments. Liver specific MRI contrast agents should not be used. To reduce potential safety concerns, macrocyclic gadolinium contrast agents are recommended per National Health Institute guidelines, or follow local standards if more rigorous.

During treatment and follow-up, radiological imaging of the chest, abdomen, pelvis, as well as all other known sites of disease, will be performed independent of treatment cycle every 6+1 weeks for the first 4 response assessments. After 4 (6 week) response assessments, radiological imaging and tumor assessment will be performed every 12+1 weeks. Radiologic imaging and tumor assessment will be performed until disease progression, start of new anti-cancer treatment, death, withdrawal of consent or until end of study. Imaging may also be performed more frequently if clinically necessitated at the discretion of the managing physician. Radiographic response (CR, PR) requires confirmation by a repeat, consecutive assessment at least 4 weeks after the first documentation of response and may be delayed until the next scheduled scan to avoid unnecessary procedures.

All subjects must have MRI of the brain performed within 28 days prior to first dose of sotorasib. Subsequently, brain scans may be performed at any time, if needed, in the judgement of the managing physician. All brain scans on protocol are required to be MRI unless MRI is contraindicated, and then CT with contrast is acceptable.

Radiological imaging assessment at the end of the study or during the end of treatment (EOT) visit should be performed only for subjects that discontinue treatment for a reason other than disease progression per RECIST 1.1 guidelines.

Determination of disease response for clinical management of subjects will be assessed at the clinical sites per RECIST 1.1. Scans will be submitted to a central imaging core laboratory for archival and independent response assessment utilizing RECIST 1.1 criteria. Exploratory imaging analyses may be performed centrally and may include tumor volumetrics, viable tumor measurements, tissue necrosis ratios, and lesion texture analysis (radiomics). Detailed information regarding submission of images to the central imaging core laboratory is found in the Site Imaging Manual.

### Adverse Events

The adverse event grading scale to be used for this study will be the CTCAE v5.0. An adverse event is any untoward medical occurrence in a clinical study subject irrespective of a causal relationship with the study treatment. Note: An adverse event can therefore be any unfavorable and unintended sign (including an abnormal laboratory finding), symptom, or disease (new or exacerbated) temporally associated with the use of a treatment, combination product, medical device or procedure.

### Events Meeting the Adverse Event Definition:

Any abnormal laboratory test results (hematology, clinical chemistry, or urinalysis) or other safety assessments (e.g., electrocardiogram, radiological scans, vital signs measurements), including those that worsen from baseline, that are considered clinically significant in the medical and scientific judgment of the investigator (i.e., not related to progression of underlying disease).

Exacerbation of a chronic or intermittent pre-existing condition including either an increase in frequency and/or intensity of the condition.

New conditions detected or diagnosed after study treatment administration even though it may have been present before the start of the study.

### Signs, Symptoms, or the Clinical Sequelae of a Suspected Drug-Drug Interaction.

Signs, symptoms, or the clinical sequelae of a suspected overdose of either study treatment or a concomitant medication. Overdose per se will not be reported as an adverse event/serious adverse event unless it is an intentional overdose taken with possible suicidal/self-harming intent. Such overdoses are to be reported regardless of sequelae.

For situations when an adverse event or serious adverse event is due to NSCLC report all known signs and symptoms. Death due to disease progression in the absence of signs and symptoms should be reported as the primary tumor type (e.g., metastatic pancreatic cancer). Note: The term “disease progression” should not be used to describe the adverse event.

“Lack of efficacy” or “failure of expected pharmacological action” per se will not be reported as an adverse event or serious adverse event. Such instances will be captured in the efficacy assessments. However, the signs, symptoms, and/or clinical sequelae resulting from lack of efficacy will be reported as adverse event or serious adverse event if they fulfill the definition of an adverse event or serious adverse event.

### Events NOT Meeting the Adverse Event Definition:

Medical or surgical procedure (e.g., endoscopy, appendectomy): the condition that leads to the procedure is the adverse event.

Situations in which an untoward medical occurrence did not occur (social and/or convenience admission to a hospital).

Anticipated day-to-day fluctuations of pre-existing disease(s) or condition(s) present or detected at the start of the study that do not worsen.

### Serious Adverse Events:

A Serious Adverse Event is defined as any untoward medical occurrence that, meets at least 1 of the following serious criteria:

### Results in Death

Immediately life threatening: The term “life-threatening” in the definition of “serious” refers to an event in which the subject was at risk of death at the time of the event. It does not refer to an event, which hypothetically might have caused death, if it were more severe.

Requires in-patient hospitalization or prolongation of existing hospitalization: In general, hospitalization signifies that the subject has been detained (usually involving at least an overnight stay) at the hospital or emergency ward for observation and/or treatment that would not have been appropriate in the physician's office or outpatient setting. Complications that occur during hospitalization are an adverse event. If a complication prolongs hospitalization or fulfills any other serious criteria, the event is serious. When in doubt as to whether “hospitalization” occurred or was necessary, the adverse event is to be considered serious. Hospitalization for elective treatment of a pre-existing condition that did not worsen from baseline is not considered an adverse event.

Results in persistent or significant disability/incapacity: The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.

### Is a Congenital Anomaly/Birth Defect

Other medically important serious event: Medical or scientific judgment is to be exercised in deciding whether serious adverse event reporting is appropriate in other situations such as important medical events that may not be immediately life-threatening or result in death or hospitalization but may jeopardize the subject or may require medical or surgical intervention to prevent 1 of the other outcomes listed in the above definition. These events are typically to be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

The investigator is responsible for ensuring that all adverse events observed by the investigator or reported by the subject that occur after the first dose of investigational product(s)/study treatment through the safety follow up are reported using the Events Case Report Form.

Since the criteria for grade 4 in the CTCAE grading scale differs from the regulatory criteria for serious adverse events, if adverse events correspond to a grade 4 CTCAE toxicity grading scale criteria (e.g., laboratory abnormality reported as grade 4 without manifestation of life-threatening status), it will be left to the investigator's judgment to also report these abnormalities as serious adverse events. For any adverse event that applies to this situation, comprehensive documentation of the event's severity must be recorded in the subject medical records.

### Response Evaluation Criteria in Solid Tumors Version 1.1 (RECIST 1.1)

### Definitions

### Measurable Lesions

Measurable Tumor Lesions—Non-nodal lesions with clear borders that can be accurately measured in at least 1 dimension with longest diameter ≥10 mm in computed tomography (CT)/magnetic resonance imaging (MRI) scan with slice thickness no greater than 5 mm. When slice thickness is greater than 5 mm, the minimum size of measurable lesion should be twice the slice thickness.

Nodal Lesions—Lymph nodes are to be considered pathologically enlarged and measurable, a lymph node must be ≥15 mm in short axis when assessed by CT/MRI (scan slice thickness recommended to be no greater than 5 mm). At baseline and in follow-up, only the short axis will be measured and followed.

Nodal size is normally reported as 2 dimensions in the axial plane. The smaller of these measures is the short axis (perpendicular to the longest axis).

Irradiated Lesions—Tumor lesions situated in a previously irradiated area, or in an area subjected to other loco-regional therapy, are not measurable unless there has been demonstrated progression in the lesion prior to enrollment.

### Non-Measurable Lesions

All other lesions, including small lesions (longest diameter <10 mm or pathological lymph nodes with ≥10 mm but to <15 mm short axis with CT scan slice thickness no greater than 5 mm) are considered non-measurable and characterized as non-target lesions.

Other examples of non-measurable lesions include:

Lesions with prior local treatment: tumor lesions situated in a previously irradiated area, or an area subject to other loco-regional therapy, should not be considered measurable unless there has been demonstrated progression in the lesion.

### Biopsied Lesions

Categorically, clusters of small lesions, bone lesions, inflammatory breast disease, and leptomeningeal disease are non-measurable.

### Methods of Measurement

Measurement of Lesions—The longest diameter of selected lesions should be measured in the plane in which the images were acquired (axial plane). All measurements should be taken and recorded in metric notation. All baseline evaluations should be performed as closely as possible to the beginning of treatment and not more than 4 weeks before study day 1.

Methods of Assessment—The same method of assessment and the same technique should be used to characterize each identified and reported lesion throughout the trial.

CT/MRI-Contrast—enhanced CT or MRI should be used to assess all lesions. Optimal visualization and measurement of metastasis in solid tumors requires consistent administration (dose and rate) of intravenous contrast as well as timing of scanning. Computed tomography and MRI should be performed with ≤5 mm thick contiguous slices.

### Baseline Documentation of “Target” and “Non-Target” Lesions

Target Lesions—All measurable lesions up to a maximum of two (2) lesions per organ and five (5) lesions in total, representative of all involved organs should be identified as target lesions and recorded and measured at baseline.

Target lesions should be selected on the basis of their size (lesions with the longest diameter) and suitability for accurate repeated measurements.

Pathologic lymph nodes (with short axis ≥15 mm) may be identified as target lesions. All other pathological nodes (those with short axis ≥10 mm but <15 mm) should be considered non-target lesions.

A sum of the diameters (longest for non-nodal lesions, short axis for nodal lesions) for all target lesions will be calculated and reported as the baseline sum of diameters. The baseline sum of diameters will be used as reference by which to characterize objective tumor response.

Non-Target Lesions—All other lesions (or sites of disease) including pathological lymph nodes should be identified as non-target lesions and should also be recorded at baseline. Measurements of these lesions are not required, and these lesions should be followed as “present”, “absent”, or “unequivocal progression” throughout the study. In addition, it is possible to record multiple non-target lesions involving the same organ as a single item on the case report form (CRF) (e.g., “multiple enlarged pelvic lymph nodes” or “multiple liver metastases”).

### Response Criteria

### Evaluation of Target Lesions

| | |
|---|---|
| Complete Response (CR): | Disappearance of all target lesions. Any pathological lymph nodes |
| | (whether target or non-target) must have reduction in short axis |
| | to <10 mm. |
| Partial Response (PR): | At least a 30% decrease in the sum of the diameters of target |
| | lesions, taking as reference the baseline sum of diameters. |
| Progressive Disease (PD): | At least a relative 20% increase and an absolute increase of 5 mm |
| | in the sum of the diameters of target lesions, taking as reference the |
| | smallest sum on study, or the appearance of one or more new |
| | lesions. |
| Stable Disease (SD) | Neither sufficient shrinkage to qualify for PR nor sufficient increase |
| | to qualify for PD, taking as reference the smallest sum of diameters |
| | since the treatment started. |

### Evaluation of Non-Target Lesions

| | |
|---|---|
| Complete | Disappearance of all non-target lesions and |
| Response (CR): | normalization of tumor marker level. All lymph |
| | nodes must be non-pathological in size |
| | (<10 mm short axis). |
| Incomplete | Persistence of one or more non-target |
| Response/Stable | lesion(s) or/and maintenance of tumor marker |
| Disease (SD): | level above the normal limits. |
| Progressive | Unequivocal progression of existing |
| Disease (PD): | non-target lesions and/or appearance of one |
| | or more new lesions.¹ |

¹To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of one or more non-target lesions is usually not sufficient to qualify for unequivocal progression status.

### Evaluation of Overall Response

The best overall response is the best response recorded from the start of the study treatment until the end of treatment or disease progression/recurrence (taking as reference for PD the smallest measurements recorded since the treatment started).

In general, the subject's best response assignment will depend on the findings of both target and non-target disease and will also take into consideration the appearance of new lesions.

Time Point response: Subjects with Target (+/−Non-target) Disease

| | | | Overall |
|---|---|---|---|
| Target Lesions | Non-target Lesions | New Lesions | Response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |

CR = complete response;
NE = Not evaluable;
PR = partial response;
PD = progressive disease;
SD = stable disease

Time Point Response: Subjects with Non-Target Disease Only

| Non-target Lesions | New Lesions | Overall Response |
|---|---|---|
| CR | No | CR |
| Non-CR/non-PD | No | Non-CR/non-PD^{‡} |
| Not all evaluated | No | NE |
| Unequivocal PD | Yes or No | PD |
| Any | Yes | PD |

^{‡}“Non-CR/non-PD” is preferred over “SD” for non-target disease since SD is increasingly used as endpoint for assessment of efficacy in some trials so to assign this category when no lesions can be measured is not advised.
CR = complete response;
NE = Not evaluable;
PD = progressive disease

### Overall Response: Confirmation of Complete Response (CR) and Partial Response (PR) Required

| Overall | Overall | |
|---|---|---|
| Response | Response | |
| First | Second | |
| Time Point | Time Point | Best Overall Response |
| CR | CF | CR |
| CR | PR | SD, PD or PR^{†} |
| CR | SD | SD provided minimum criteria for |
| | | SD duration met, otherwise, PD |
| CR | PC | SD provided minimum criteria for |
| | | SD duration met, otherwise, PD |
| CR | NE | SD provided minimum criteria for |
| | | SD duration met, otherwise, NE |
| PR | CR | PR |
| PR | PR | PR |
| PR | SD | SD |
| PR | PD | SD provided minimum criteria for |
| | | SD duration met, otherwise, PD |
| PR | NE | SD provided minimum criteria for |
| | | SD duration met, otherwise, NE |
| NE | NE | NE |

^{†}If a CR is truly met at first time point, then any disease at a subsequent time point, even disease meeting PR criteria relative to baseline, makes the disease PD at that point (since disease must have reappeared after CR). Best response would depend on whether minimum duration for SD was met. However, sometimes “CR” may be claimed when subsequent scans suggest small lesions were likely still present and in fact the subject had PR, not CR at the first time point. Under these circumstances, the original CR should be changed to PR and the best response is PR.
NE = Not evaluable;
PR = partial response

### Special Notes on Response Assessment

Nodal lesions—Lymph nodes identified as target lesions should always have the actual short axis measurement recorded, even if the nodes regress to below 10 mm on study. In order to qualify for complete response (CR), each node must achieve a short axis <10 mm, NOT total disappearance. Nodal target lesion short axis measurements are added together with target lesion’ longest diameter measurements to create the sum of target lesion diameters for a particular assessment (timepoint).

Target lesions that become “too small to measure”—While on study, all lesions (nodal and non-nodal) recorded at baseline should have their measurements recorded at each subsequent evaluation. If a lesion becomes less than 5 mm, the accuracy of the measurement becomes reduced. Therefore, lesions less than 5 mm are considered as being “too small to measure”, and are not measured. With this designation, they are assigned a default measurement of 5 mm. No lesion measurement less than 5 mm should be recorded, unless a lesion totally disappears and “0” can be recorded for the measurement.

New lesions—The term “new lesion” always refers to the presence of a new finding that is definitely tumor. New findings that only may be tumor, but may be benign (infection, inflammation, etc.) are not selected as new lesions, until that time when the review is certain they represent tumor.

If a new lesion is equivocal, for example because of its small size, continued therapy and follow-up evaluation will clarify if it represents truly new disease. If repeat scans confirm there is definitely a new lesion, then progression should be declared using the date of the initial scan.

A lesion identified on a follow-up study in an anatomical location that was not scanned at baseline is considered a new lesion and will indicate disease progression, regardless of any response that may be seen in target or non-target lesions present from baseline.

Subjects with a global deterioration of health status requiring discontinuation of treatment without objective evidence of disease progression at that time should be classified as having “symptomatic deterioration.” Every effort should be made to document the objective progression with an additional imaging assessment even after discontinuation of treatment.

In some circumstances it may be difficult to distinguish residual disease from scar or normal tissue. When the evaluation of CR depends on this determination, it is recommended that the residual lesion be further investigated by fluorodeoxyglucose-positron emission tomography (FDG/PET) or PET/CT, or possibly fine needle aspirate/biopsy, to confirm the CR status.

### Confirmation Measurement/Duration of Response

Response Confirmation-In non-randomized trials where response is the primary endpoint, confirmation of partial response (PR) and CR is required to ensure responses identified are not the result of measurement error.

Duration of overall response—The duration of overall response is measured from the time measurement criteria are first met for CR/PR (whichever is first recorded) until the first date the recurrent or progressive disease is objectively documented.

Duration of Stable Disease—Stable disease (SD) is measured from the start of the treatment until the criteria for disease progression are met, taking as reference the smallest measurements recorded since the treatment started.

### ECOG Performance and NYHA Classification

ECOG Performance Status Scale

| Grade | Descriptions |
|---|---|
| 0 | Fully active, able to carry on all pre-disease |
| | performance without restriction. |
| 1 | Restricted in physically strenuous activity, but |
| | ambulatory and able to carry out work of a |
| | light or sedentary nature (e.g., light |
| | housework, office work). |
| 2 | Ambulatory and capable of all self-care, but unable |
| | to carry out any work activities. Up and |
| | about more than 50% of waking hours. |
| 3 | Capable of only limited self-care, confined to bed |
| | or chair more than 50% of waking hours. |
| 4 | Completely disabled. Cannot carry on any self-care. |
| | Totally confined to bed or chair. |
| 5 | Dead. |

### Source: Oken et al, 1982; ECOG=Eastern Cooperative Oncology Group; NYHA=New York Heart Association

### New York Heart Association Functional Classification

Class I No limitation of physical activity. Ordinary physical activity does not cause undue fatigue, palpitation or dyspnea.
Class II Slight limitation of physical activity. Comfortable at rest, but ordinary physical activity results in fatigue, palpitation or dyspnea.
Class III Marked limitation of physical activity. Comfortable at rest, but less than ordinary activity causes fatigue, palpitation or dyspnea.
Class IV Unable to carry out any physical activity without discomfort. Symptoms of cardiac insufficiency may be present even at rest. If any physical activity is undertaken, discomfort is increased.

### Example 2

KRAS^{G12C} inhibitor-naïve patients (n-58) with KRAS G12C-mutated NSCLC were treated in 12 dose exploration cohorts at varying doses of sotorasib (120 mg, 240 mg, 360 mg, 720 mg, and 960 mg QD) in combination with either intravenous atezolizumab 1200 mg or pembrolizumab 200 mg, administered concurrently every 3 weeks until intolerability or disease progression. (CodeBreak 100 (ClinicalTrials.gov Identifier: NCT03600883) and CodeBreak 101 (ClinicalTrials.gov Identifier: NCT04185883) Phase 1b dose exploration). The data presented herein, at least in part, was subject to a presentation at the 2022 World Conference on Lung Cancer in Vienna, Austria (Aug. 6-9, 2022) by Li et al. entited “CodeBreak 100/101: First report of safety and efficacy of sotorasib in combination with pembrolizumab or atezolizumab in advanced KRAS p.G12C NSCLC,” abstract and slides, which are incorporated herein by reference in their entirety.

Baseline characteristics of the 58 patients are shown in Table 11.

TABLE 11

| | | |
|---|---|---|
| Median age, years (range) | 66 | (29, 86) |
| Median prior lines of therapy, n (range) | 1.0 | (0, 7) |
| Treated as first line therapy, n (%) | 12 | (21) |
| Prior anti-PD-L1, n (%) | 39 | (67) |
| Best overall response of PD, n (%) | 20/39 | (51) |
| ECOG performance score, n (%) | | |
| 0 | 11 | (19.0) |
| 1 | 47 | (81.0) |
| Brain metastasis, n (%) | 18 | (31) |
| Liver metastasis, n (%) | 15 | (26) |
| PD-L1 expression, n (%)* | | |
| <1% | 10 | (17) |
| 1% to 49% | 16 | (28) |
| ≥50% | 21 | (36) |
| Smoking history, n (%)^{#} | 54 | (93) |

*not available for all patients
^{#}Includes former and current smoking history

ECOG, Eastern Cooperative Oncology Group; PD, progressive disease; PD-L1, programmed death ligand 1

Two of the cohorts were lead-in cohorts where patients received sotorasib monotherapy for either 21 or 42 days prior to their first dose of immunotherapy (IO), then received Atezo or Pembro together with sotorasib. Primary objective was safety/tolerability; secondary efficacy objectives included ORR (objective response rate, investigator assessment per RECIST v1.1) and disease control rate (DCR). The dose limiting toxicity (DLT) window was 21 days following initiation of combination treatment.

### Results

Safety: 58 patients were treated with a median follow-up of 12.8 months (range: 1.6, 29.9). Median prior lines of therapy were 1 (range 0-7); 67% patients received prior IO. The median doses of sotorasib were 83 [range: 22-791], and median doses of IO were 3 [range: 1-26]). The most common grade 3-4 treatment-related adverse events (TRAEs) were increased ALT and AST (Table 12). Of patients with grade 3-4 hepatotoxicity TRAEs, first occurrence was outside the DLT window in 22 of 25 (88%) patients, most were managed with corticosteroids, and 97% of events resolved. No fatal TRAEs occurred. Grade 3-4 TRAEs and TRAEs leading to treatment discontinuation occurred less often in the lead-in versus concurrent cohorts (Table 12).

TABLE 12

| | | | |
|---|---|---|---|
| Soto + | Soto + | Soto + | Soto + |
| Atezo Soto | Atezo | Pembro Soto | Pembro |
| Lead-In | Concurrent | Lead-In | Concurrent |
| (N = 10) | (N = 10) | (N = 19) | (N = 19) |

Safety, n (%)

| TRAE, any grade | 10 | (100) | 9 | (90) | 15 | (79) | 17 | (89) |
|---|---|---|---|---|---|---|---|---|
| TRAE, grade 3 | 3 | (30) | 5 | (50) | 10 | (53) | 14 | (74) |
| TRAE, grade 4^{#} | 0 | | 1 | (10) | 0 | | 1 | (5) |
| Hepatotoxicity, ≥3^{a}, N1 | 3 | (30) | 5 | (50) | 8 | (42) | 9 | (47) |
| ALT increased | 1 | (10) | 4 | (40) | 6 | (32) | 7 | (37) |
| AST increased | 0 | | 5 | (50) | 5 | (26) | 5 | (26) |
| Median time to | 50 | (28, 93) | 67 | (36, 147) | 72 | (45, 127) | 51 | (29, 190) |
| onset, days (range) | | | | | | | | |
| Events first | 2/3 | (67) | 5/5 | (100) | 6/8 | (75) | 9/9 | (100) |
| occurring outside | | | | | | | | |
| DLT window* n/N1 (%) | | | | | | | | |
| TRAE leading to | 1 | (10) | 5 | (50) | 6 | (32) | 10 | (53) |
| discontinuation of | | | | | | | | |
| Sotorasib and/or IO | | | | | | | | |

Number of treatment doses, median (min, max)

| | | | | | | | | |
|---|---|---|---|---|---|---|---|---|
| Sotorasib | 129.5 | (36, 562) | 115.0 | (22, 422) | 73.0 | (41, 426) | 82.0 | (35, 791) |
| IO | 2.5 | (1, 26) | 3.5 | (2, 21) | 1.0 | (1, 20) | 3.0 | (2, 12) |

Efficacy

| ORR, % (95% CI) | 20 | (3, 56) | 20^{b} | (3, 56) | 37^{c} | (16, 62) | 32^{d} | (13, 57) |
|---|---|---|---|---|---|---|---|---|
| DCR, % (95% CI) | 90 | (56, 100) | 80 | (44, 98) | 74 | (49, 91) | 90 | (67, 99) |
| Median DpR, % | 43% | | 43% | | 63% | | 66% | |
| Median OS, (95% CI), | 8.1 | (2.5, NE) | 11.5 | (5.0, NE) | NE | (10.1, NE) | 14.1 | (6.2, 17.8) |
| months | | | | | | | | |

TRAE—treatment-related adverse event; N1—indicates number of grade ≥3 treatment related hepatotoxicity; ALT—alanine aminotransferase; AST—aspartate aminotransferase; DLT—dose limiting toxicity; IO—immune-oncology therapy (i.e., azetolizumab or pembrolizumab); ORR—objective response rate; DCR, DpR—median depth of response, OS—overall survival; NE—not established.
*DLT window was 21 days following initiation of combination treatment of sotorasib and IO.
^{#}Grade 4 TRAE were all ALT increase (n = 1) and AST increase (n = 1).
^{a}No suspected drug-induced liver injury cases by Hy's law occurred. Hepatic Disorders broad search terms include ALT increased, AST increased, immune-mediated hepatitis, blood alkaline phosphatase increased, blood bilirubin increased, and gamma glutamyltransferase increased. For Soto + Pembro with Soto Lead-in, term hepatic enzyme increased was also included. For Soto + Atezo cohorts, terms also included liver function test increased, drug-induced liver injury, and transaminases increased.
^{b}Combined efficacy of 3 dose cohorts
^{c}Combined efficacy of 5 dose cohorts
^{d}Combined efficacy of 3 dose cohorts

The most common TRAEs from the study are shown in the Table 13 below.

TABLE 13

| | Sotorasib + | | Sotorasib + | | Sotorasib + | | Sotorasib + | |
|---|---|---|---|---|---|---|---|---|
| | Atezolizumab | | Atezolizumab | | Pembrolizumab | | Pembrolizumab | |
| | Lead-In | | Concurrent | | Lead-In | | Concurrent | |
| TRAEs, n (%) | (N = 10) | | (N = 10) | | (N = 19) | | (N = 19) | |
| All grades | 10 | (100) | 9 | (90) | 15 | (79) | 17 | (89) |
| Diarrhea | 6 | (60) | 8 | (80) | 11 | (58) | 13 | (68) |
| ALT and AST | 3 | (30) | 6 | (60) | 9 | (47) | 12 | (63) |
| increased | | | | | | | | |
| Fatigue | 4 | (40) | 2 | (20) | 5 | (26) | 11 | (58) |
| Nausea | 5 | (50) | 2 | (20) | 9 | (47) | 10 | (53) |
| Grades 3-4 | 3 | (30) | 6 | (60) | 10 | (53) | 15 | (79) |
| ALT increased | 1 | (10) | 4 | (40) | 6 | (32) | 7 | (37) |
| AST increased | 0 | | 5 | (50) | 5 | (26) | 5 | (26) |
| Colitis | 0 | | 0 | | 0 | | 3 | (16) |
| Blood-alkaline | 1 | (10) | 2 | (20) | 2 | (11) | 4 | (21) |
| phosphatase | | | | | | | | |
| increased | | | | | | | | |

| | | | | | | | |
|---|---|---|---|---|---|---|---|
| Diarrhea | 0 | 2 | (20) | 1 | (5) | 1 | (5) |

Efficacy: Across all 12 cohorts, confirmed response was observed in 17 of 58 patients (29%; range, 0-67). The ORR for IO-naive was 26% (5 PRs out of 19 patients). The ORR for IO-exposed was 31% (2 CRs, 10 PRs out of 39 patients). Among the 17 responders, median duration of response was 17.9 months (range: 1.5+, 23.4). 5 out of 17 patients had observed a duration of response (DOR) great than 10 month with 8 ongoing responders. For all 58 patients across cohorts, median OS was 15.7 months (95% CI: 9.8, 17.8). A response was observed irrespective of PD-L1 expression levels (TPS unknown (DCR=91%; ORR=27%, Median Depth of Response (Median DpR)=62%); TPS less than 1% (DCR=100%; ORR=50%; Median DpR 51%; TPS 1% to 49% (DCR=75%; ORR=19%; Median DpR=73%); TPS 50% or greater (DCR=76%; ORR=29%; Median DpR=45%). Further, a response was observed irrespective of sotorasib dose.

Cohorts having a lead-in therapy demonstrated durable clinical activity with lower rates of grade 3-4 TRAEs and TRAEs leading to discontinuation, compared to cohorts without a lead-in.

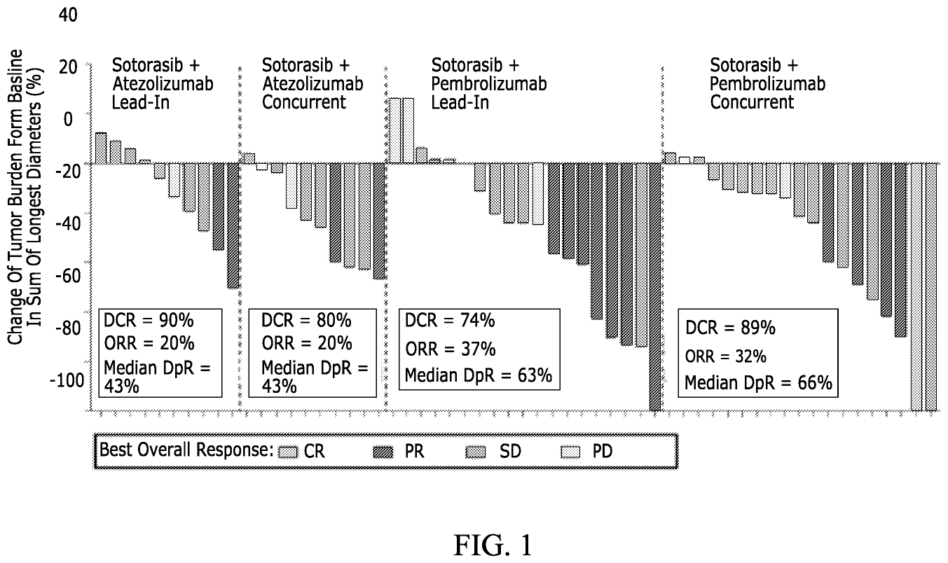

Efficacy data for the four cohorts (sotorasib and azetolizumab or pembrolizumab, with or without a lead-in monotherapy with sotorasib) are shown in FIG. 1. The efficacy data show that there was a greater depth of response and higher efficacy for the sotorasib and pembrolizumab therapy, compared with the sotorasib and azetolizumab therapy, with or without the lead-in monotherapy with sotorasib.

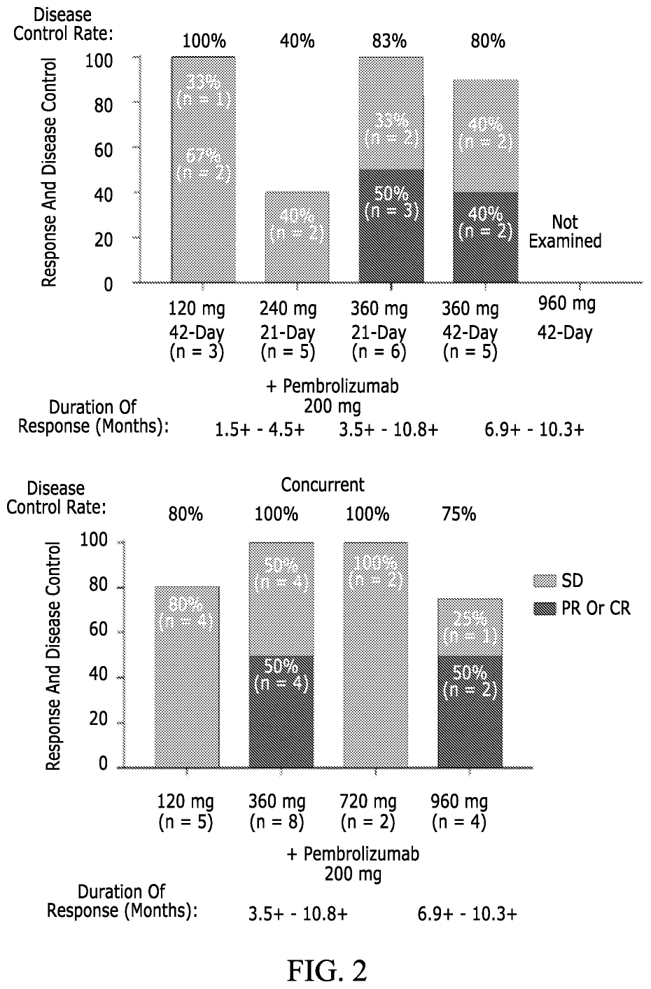

The efficacy of sotorasib at the 4 doses tested (120, 240, 360, and 960) with 200 mg pembrolizumab, with or without a lead-in sotorasib monotherapy, is shown in FIG. 2.

### CONCLUSIONS

In this mostly IO-pretreated NSCLC population, sotorasib with azetolizumab or pembrolizumab led to a higher incidence of grade 3-4 TRAEs than observed with monotherapy. Grade 3-4 TRAEs were primarily liver enzyme elevations, with nearly all events occurring outside the DLT window and 97% of events resolved. Lead-in cohorts demonstrated durable clinical activity with lower rates of grade 3-4 TRAEs and TRAEs leasing to discontinuation compared to concurrent cohorts. Five of 17 patients had observed DOR greater than 10 months with 8 ongoing responders. Dose expansion is ongoing in treatment-naïve patients using sotorasib lead-in followed by combination of sotorasib with pembrolizumab.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### REFERENCES

Ahn et al, J Thorac Oncol. 2016; 11 (4): S115.
Albert et al. Nat. Methods 2007; 4:903-905.
The American Association for Cancer Research [AACR] Project GENIE Consortium, 2017 https://www.aacr.org/RESEARCH/RESEARCH/PAGES/AACR-PROJECT-GENIE.ASPX.
BAVENCIO® U.S. Prescribing Information, EMD Serono, Inc., Rockland, Maryland, 02370 (revision 11/2020)
Beers and Nederlof, Breast Cancer Res. 2006; 8 (3): 210.
Bertone et al. Genome Res 2006; 16 (2): 271-281.
Biernacka et al, Cancer Genet. 2016; 209 (5): 195-198.
Borghaei and Brahmer, N Engl J Med. 2016; 374 (5): 493-494.
Canon et al, Nature. 2019; 575 (7781): 217-223.
Cecil, Textbook of Medicine, 1985; W.B. Saunders & Co., 2317-2341.
Cerami et al., Cancer Discov. 2012, 2 (5), 401.
Chen et al, Cancer Immunol Immunother. 2017; 66:1175-1187.
Chung et al. Genome Res. 2004; 14 (1): 188-196.
Cully and Downward, Cell. 2008; 133:1292.
Dalma-Weiszhausz et al. Methods Enzymol. 2006; 410:3-28.
DeRisi et al., Nat. Genet. 1996; 14:457-460.
Eisenhauer et al., Eur J Cancer, 2009; 45 (2): 228-247.
www.fda.gov/drugs/drug-interactions-labeling/drug-development-and-drug-interactions-table-substrates-inhibitors-and-inducers, accessed May 2021.
Flockhart D A, Drug Interactions: Cytochrome P450 Drug Interaction Table. Indiana University School of Medicine (2007), www.drug-interactions.medicine.iu.edu, accessed May 2021.
Forshew et al., Sci Transl Med; 2012; 4: 136ra68.
Gao et al., Science Signaling 2013, 6 (269), pl1.
Gibbons et al, J Thorac Oncol. 2016; 11 (4): S79.
Guidance for Industry Drug-Induced Liver Injury: Premarketing Clinical Evaluation, July 2009.
Guo et al, Contemp Clin Trials. 2017; 58:23-33.
Haber and Velculescu, Cancer Discov., 2014; 4:650-61.
Herbst et al, Annals of Oncology. 2019; 30: Supplement 11.
Hong et al., N Engl J Med. 2020; 383 (13): 1207-1217.
Hughes et al. Nat. Biotechnol. 2001; 19 (4): 342-347.
IMFINZI® U.S. Prescribing Information, AstraZeneca Pharmaceuticals LP, Wilmington, Delaware, 19850 (revision July 2021).
Irizarry Nucleic Acids Res 2003, 31: e15.
Janes et al., Cell. 2018; 172 (3): 578-589.
Jasmine et al. PLoS One 2012; 7 (2): e31968.
KEYTRUDA® U.S. Prescribing Information, Merck & Co., Inc., Whitehouse Station, New Jersey, 08889 (revision February 2022).
Kim et al. Carcinogenesis 2006; 27 (3): 392-404.
Kinde et al., Proc Natl Acad Sci USA; 2011, 108:9530-5.
Kumar et al. J. Pharm. Bioallied Sci. 2012; 4 (1): 21-26.
Laere et al. Methods Mol. Biol. 2009; 512:71-98.
Lanman et al., J. Med. Chem. 2020, 63, 52.
Lee et al, JAMA Oncol. 2018; 4 (2): 210-216.
Li et al.; “CodeBreak 100/101: First report of safety and efficacy of sotorasib in combination with pembrolizumab or atezolizumab in advanced KRAS p.G12C NSCLC” World Conference on Lung Cancer, 2022 (August 6-9); Vienna Austria.
Lin et al. BMC Genomics 2010; 11:712.
Liu et al. Biosens Bioelectron 2017; 92:596-601.
Lodes et al. PLoS One 2009; 4 (7): e6229.
LUMAKRAS® US Prescribing Information, Amgen Inc., Thousand Oaks, California, 91320 (revision May 2021).
Mackay et al. Oncogene 2003; 22:2680-2688.
Mao et al. Curr. Genomics 2007; 8 (4): 219-228.
Mazieres et al, Ann Oncol. 2019; 30 (8): 1321-1328.
McDonald et al., Cell. 2017; 170 (3): 577-592.
Michels et al. Genet. Med. 2007; 9:574-584.
Mockler and Ecker, Genomics 2005; 85 (1): 1-15.
National Cancer Institute Common Terminology Criteria for Adverse Events v5.0 (NCI CTCAE) published Nov. 27, 2017.
Neumann et al, Pathol Res Pract. 2009; 205 (12): 858-862.
Oken et al, 1 Am J Clin Oncol. 1982; 5 (6): 649-655.
Ostrem et al., Nature. 2013; 503 (7477): 548-551.
Ostrem and Shokat, Nat Rev Drug Discov. 2016; 15 (11): 771-785.
Patricelli et al., Cancer Discov. 2016; 6 (3): 316-329.
Pinkel et al. Nat. Genetics 2005; 37: S11-S17.
Ribas et al, N Eng J Med. 2013; 368 (14): 1365-1366.
Rini et al, N Engl J Med. 2019; 380:1116-1127.
Simanshu et al., Cell. 2017; 170 (1): 17-33.
Sullivan et al, Nat Med. 2019; 25 (6): 929-935.
TECENTRIQ® U.S. Prescribing Information, Genentech, Inc., South San Francisco, California, 94080 (revision January 2022).
Thomas et al. Genome Res. 2005; 15 (12): 1831-1837.
Thompson et al., PLoS ONE, 2012; 7: e31597.
Wang et al. Cancer Genet 2012; 205 (7-8): 341-55.
Wei et al. Nucleic Acids Res 2008; 36 (9): 2926-2938.
Xie et al., Front Pharmacol.; 2017; 8:823.
Yang et al, J Thorac Oncol. 2019; 14 (3): 553-559.
Zubrod et al., Chronic Disease, 1960; 11:7-33.

What is claimed is:

1. A method of treating cancer comprising a KRAS G12C mutation in a patient comprising
(a) administering to the patient a therapeutically effective amount of sotorasib for 14 to 48 days (“an induction period”), and
(b) administering to the patient a therapeutically effective amount of sotorasib and a therapeutically effective amount of an anti-PD1 antibody or an anti-PD-L1 antibody after the induction period for the duration of a combination period.

2. The method of claim 1, wherein the therapeutically effective amount of sotorasib administered for the duration of the induction period is 960 mg.

3. The method of claim 1, wherein the therapeutically effective amount of sotorasib administered during the induction period is 360 mg.

4. The method of claim 1, wherein the therapeutically effective amount of sotorasib administered during the induction period is 240 mg.

5. The method of claim 1, wherein the therapeutically effective amount of sotorasib administered during the induction period is 120 mg.

6. The method of any one of claims 1 to 5, comprising administering the therapeutically effective amount of sotorasib to the patient once daily during the induction period.

7. The method of any one of claims 1 to 5, comprising administering the therapeutically effective amount of sotorasib to the patient twice daily during the induction period, wherein each dose of sotorasib corresponds to half of the therapeutically effective amount administered during the induction period.

8. The method of any one of claims 1 to 7, wherein the therapeutically effective amount of sotorasib administered during the combination period is 960 mg.

9. The method of any one of claims 1 to 7, wherein the therapeutically effective amount of sotorasib administered during the combination period is 360 mg.

10. The method of any one of claims 1 to 7, wherein the therapeutically effective amount of sotorasib administered during the combination period is 240 mg.

11. The method of any one of claims 1 to 7, wherein the therapeutically effective amount of sotorasib administered during the combination period is 120 mg.

12. The method of any one of claims 1 to 11, comprising administering the therapeutically effective amount of sotorasib to the patient once daily during the combination period.

13. The method of any one of claims 1 to 11, comprising administering the therapeutically effective amount of sotorasib to the patient twice daily during the combination period, wherein each dose of sotorasib corresponds to half of the therapeutically effective amount administered during the combination period.

14. The method of any one of claims 1 to 13, wherein the anti-PD-L1 antibody is atezolizumab, avelumab, or durvalumab.

15. The method of claim 14, wherein the anti-PD-L1 antibody is atezolizumab.

16. The method of any one of claims 1 to 13, wherein the anti-PD1 antibody is cemiplimab, dostarlimab, pembrolizumab, or nivolumab.

17. The method of claim 16, wherein the anti-PD1 antibody is pembrolizumab.

18. The method of claim 17, comprising administering to the patient 200 mg pembrolizumab via IV once every three weeks during the combination period.

19. The method of claim 1, comprising administering to the patient
360 mg sotorasib orally once per day during the induction period and the combination period; and
200 mg pembrolizumab via IV once every three weeks during the combination period.

20. The method of claim 1, comprising administering to the patient
960 mg sotorasib orally once per day during the induction period and the combination period; and
200 mg pembrolizumab via IV once every three weeks during the combination period.

21. The method of claim 1, comprising administering to the patient
240 mg sotorasib orally once per day during the induction period and the combination period; and
200 mg pembrolizumab via IV once every three weeks during the combination period.

22. The method of claim 1, comprising administering to the patient
120 mg sotorasib orally once per day during the induction period and the combination period and
200 mg pembrolizumab via IV once every three weeks during the combination period.

23. The method of any one of claims 1 to 22, wherein the induction period is 21 days.

24. The method of any one of claims 1 to 22, wherein the induction period is 42 days.

25. The method of any one of claims 1 to 24, wherein the combination period is at least 30 days.

26. The method of claim 25, wherein the combination period is at least 3 months.

27. The method of claim 26, wherein the combination period is at least 6 months.

28. The method of claim 27, wherein the combination period is at least 8 months.

29. The method of any one of claims 1 to 28, wherein the cancer exhibits a PD-L1 tumor proportion score (TPS) of 1% or greater.

30. The method of any one of claims 1 to 28, wherein the cancer exhibits a PD-L1 tumor proportion score (TPS) of 50% or greater.

31. The method of any one of claims 1 to 28, wherein the cancer exhibits a PD-L1 tumor proportion score (TPS) of 1% to 49%.

32. The method of any one of claims 1 to 28, wherein the cancer exhibits a PD-L1 tumor proportion score (TPS) of less than 1%.

33. The method of any one of claims 1 to 32, wherein the cancer is a solid tumor.

34. The method of any one of claims 1 to 32, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

35. The method of any one of claims 1 to 32, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

36. The method of any one of claims 1 to 32, wherein the cancer is non-small cell lung cancer.

37. The method of claim 36, wherein the cancer is locally-advanced or metastatic non-small cell lung cancer.

38. The method of any one of claims 1 to 37, wherein the patient exhibits at least a stable disease (SD), as measured by RECIST 1.1 protocol, after the combination period lasts 3, 6, or 8 months.

39. The method of any one of claims 1 to 37, wherein the patient exhibits at least a partial response (PR), as measured by RECIST 1.1 protocol, after the combination period lasts 3, 6, or 8 months.

40. The method of any one of claims 1 to 37, wherein the patient exhibits a progression free survival (PFS) of at least 3 months.

41. The method of any one of claims 1 to 40, wherein the patient exhibits fewer grade 3 or 4 treatment related adverse events (TRAEs) compared to a patient administered sotorasib and the anti-PD1 antibody or anti-PD-L1 antibody without an induction period.

42. The method of any one of claims 1 to 41, wherein the patient has not received any prior line of therapy.

43. The method of any one of claims 1 to 41, wherein the patient has received at least one prior line of therapy.

44. The method of any one of claims 1 to 43, wherein the patient has not previously received treatment with an anti-PD1 or anti-PD-L1 immunotherapy.

45. The method of any one of claims 1 to 43, wherein the patient has previously received treatment with anti-PD1 or anti-PD-L1 immunotherapy.

46. The method of any one of claims 1 to 41 and 43, wherein the patient has previously received treatment with (i) anti-PD1 or anti-PDL1 immunotherapy or (ii) prior platinum-based combination chemotherapy.

47. The method of any one of claims 1 to 41 and 43, wherein the patient has previous received treatment with (i) anti-PD1 or anti-PD-L1 immunotherapy and (ii) prior platinum-based combination chemotherapy.

48. The method of any one of claims 1 to 41 and 43 to 46, wherein the patient has previously undergone an EGFR, ALK or ROS1 targeted therapy if the cancer also exhibited a mutation in EGFR, ALK, or ROS1.

49. The method of claim 48, wherein the patient has progressed on an EGFR, ALK or ROS1 targeted therapy if the cancer also exhibited a mutation in EGFR, ALK, or ROS1.

50. The method of any one of claims 43 to 49, wherein the patient completed neoadjuvant or adjuvant chemotherapy at least 12 months prior to diagnosis of advanced stage cancer.

51. The method of claim any one of claims 1 to 41, wherein
(1) (a) the patient has previously received treatment with (i) anti-PD1 or anti-PD-L1 immunotherapy or (ii) prior platinum-based combination chemotherapy; or (b) the patient has previously received treatment with (i) anti-PD1 or anti-PD-L1 therapy and (ii) prior platinum-based chemotherapy; and
(2) the patient optionally has previously undergone an EGFR, ALK or ROS1 targeted therapy if the cancer also exhibited a mutation in EGFR, ALK, or ROS1.

52. The method of any one of claims 1 to 41, wherein the patient
(1) has a cancer that exhibits a PD-L1 tumor proportion score (TPS) of 50% or greater; and
(2) has not received any systemic therapy for locally advanced or metastatic non-small cell lung cancer;
(i) but for a EGFR, ALK, or ROS1 targeted cancer therapy, if cancer exhibited a mutation in EGFR, ALK, or ROS1, and the patient has progressed on the targeted cancer therapy; and
(ii) but for neoadjuvant or adjuvant chemotherapy completed at least 12 months prior to the start of the induction period and has not received immune checkpoint inhibitor therapy.

53. The method of any one of claims 1 to 41, wherein the patient
(1) has a cancer that exhibits a PD-L1 tumor proportion score (TPS) of 1% or greater; and
(2) has not received any systemic therapy for locally advanced or metastatic non-small cell lung cancer;
(i) but for a EGFR, ALK, or ROS1 targeted cancer therapy, if cancer exhibited a mutation in EGFR, ALK, or ROS1, and the patient has progressed on the targeted cancer therapy; and
(ii) but for neoadjuvant or adjuvant chemotherapy completed at least 12 months prior to the start of the induction period and has not received immune checkpoint inhibitor therapy.

54. The method of any one of claims 1 to 53, wherein the patient has an Eastern Cooperative Oncology Group (ECOG) performance status of less than or equal to 2.

55. The method of any one of claims 1 to 54, wherein the patient does not have active (symptomatic) brain metastases.

56. The method of any one of claims 1 to 55, wherein
(i) the patient
had brain metastases resected, or
received whole brain radiation therapy ending at least 4 weeks prior to start of the induction period, or
received stereotactic radiosurgery ending at least 2 weeks prior to start of the induction period, and
(ii) the patient
exhibits residual neurological symptoms of grade 2 or less, and
has not been administered steroids for at least 14 days prior to the start of the induction period, and
has an magnetic resonance imaging (MRI) performed within 14 days prior to start of the induction period that shows no evidence of progression of the brain metastases.

57. The method of any one of claims 1 to 56, wherein the patient does not have leptomeningeal disease.

58. The method of any one of claims 1 to 57, wherein the patient is not suffering from a hepatitis B infection or a hepatitis C infection.

59. The method of any one of claims 1 to 58, wherein the patient has not received a prior therapy with a KRAS^{G12C} inhibitor.

60. The method of claim 59, wherein the KRAS^{G12C} inhibitor is sotorasib or adagrasib.

61. The method of any one of claims 1 to 60, wherein the patient is in further need of treatment with an acid-reducing agent.

62. The method of claim 61, wherein the acid-reducing agent is a proton pump inhibitor (PPI), a H2 receptor antagonist (H2RA), or a locally acting antacid.

63. The method of claim 61 or claim 62, wherein the acid-reducing agent is a locally acting antacid, and wherein sotorasib is administered about 4 hours before or about 10 hours after the locally acting antacid.

64. The method of claim 62 or 63, wherein the locally acting antacid is sodium bicarbonate, calcium carbonate, aluminum hydroxide, or magnesium hydroxide.

65. The method of any one of claims 62 to 64, wherein the patient is in further need of treatment with a proton pump inhibitor (PPI) or H2 receptor antagonist (H2RA).

66. The method of claim 65, wherein the patient is not administered a PPI or a H2RA in combination with sotorasib.

67. The method of any one of claim 62, 65 or 66, wherein the PPI is omeprazole, pantoprazole, esomeprazole, lansoprazole, rabeprazole, or dexlansoprazole.

68. The method of any one of claim 62, 65 or 66, wherein the H2RA is famotidine, ranitidine, cimetidine, nizatidine, roxatidine or lafutidine.

69. The method of any one of claims 1 to 68, wherein the patient is in further need of treatment with a CYP3A4 inducer.

70. The method of claim 69, wherein the patient is not administered a CYP3A4 inducer in combination with sotorasib.

71. The method of claim 69 or 70, wherein the CYP3A4 inducer is a apalutamide, avasimibe, barbiturate, brigatinib, carbamazepine, clobazam, dabrafenib, efavirenz, elagolix, enzalutamide, eslicarbazepine, glucocorticoids, ivosidenib, letermovir, lorlatinib, lumacaftor, mitotane, modafinil, nevirapine, oritavancin, oxcarbazepine, perampanel, phenobarbital, phenytoin, pioglitazone, rifabutin, rifampin, rifapentine, St. John's wort, telotristat, or troglitazone.

72. The method of claim 69 or 70, wherein the CYP3A4 inducer is a strong CYP3A4 inducer.

73. The method of claim 72, wherein the strong CYP3A4 inducer is rifampin, mitotane, avasimibe, rifapentine, apalutamide, ivosidenib, phenytoin, carbamazepine, enzalutamide, St John's Wort extract, or lumacaftor.

74. The method of any one of claims 1 to 73, wherein the patient is in further need of treatment with a CYP3A4 substrate.

75. The method of claim 74, wherein the patient is not administered a CYP3A4 substrate in combination with sotorasib.

76. The method of claim 72 or 73, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus (fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, or ziprasidone.

77. The method of claim 74 or 75, wherein the CYP3A4 substrate is a CYP3A4 substrate with a narrow therapeutic index.

78. The method of claim 77, wherein the CYP3A4 substrate with a narrow therapeutic index is alfentanil, cyclosporine, dihydroergotamine, ergotamine, everolimus, fentanyl, primozide, quinidine, tacrolimus, or sirolimus.

79. The method of any one of claims 1 to 78, wherein the patient is in further need of treatment with a P-glycoprotein (P-gp) substrate.

80. The method of claim 79, wherein the patient is not administered a P-gp substrate in combination sotorasib.

81. The method of claim 79 or 80, wherein the P-gp substrate is etexilate, digoxin, fexofenadine, everolimus, cyclosporine, sirolimus, tacrolimus, or vincristine.

82. The method of claim 79 or 80, wherein the P-gp substrate is a P-gp substrate with a narrow therapeutic index.

83. The method of claim 82, where in the P-gp substrate with a narrow therapeutic index is digoxin, everolimus, cyclosporine, tacrolimus, sirolimus, or vincristine.
